(19) United States
(12) Patent Application Publication
(10) Pub. No.: US 2025/0064782 A1
(43) Pub. Date: Feb. 27, 2025

(54) Methods of Treating Substance Use Disorders Using 5-MeO-DMT

(71) Applicant: Journey Colab Corp., San Francisco, CA (US)

(72) Inventors: Jeeshan Chowdhury, San Francisco, CA (US); Monica Alfaro Welling, Whittier, CA (US)

(21) Appl. No.: 18/720,892

(22) PCT Filed: Dec. 19, 2022
(86) PCT No.: PCT/US22/53428

§ 371 Date: Jun. 17, 2024

Related U.S. Application Data
(60) Provisional application No. 63/291,210, filed on Dec. 17, 2021.

(51) Int. Cl. A61K 31/4045

(52) U.S. Cl. CPC A61K 31/4045

(57) ABSTRACT

Disclosed are methods of treating substance use disorders with formulations comprising 5-MeO-DMT or a salt thereof. such as by intravenous, intramuscular, or buccal administration. In some embodiments, treatment comprises one or more therapy sessions. Methods further comprise a plurality of treatment steps to aid in the efficacy of treating moderate to severe substance use disorders.

### CROSS-REFERENCE

Priority is claimed under PCT Art. 8 (1) and Rule 4.10 to U.S. Prov. Appl. No. 63/291,210, filed Dec. 17, 2021, and incorporated by reference for all purposes as if fully set forth herein.

### FIELD OF THE INVENTION

This disclosure relates in some aspects to using 5-methoxy-N,N-dimethyltryptamine (5-MeO-DMT) to treat substance use disorders (SUDs). In some aspects, 5-MeO-DMT is administered in conjunction with psychotherapy. In some aspects, this disclosure further relates to methods of synthesizing 5-MeO-DMT, compositions comprising 5-MeO-DMT, and methods of using such compositions, including routes of administration and use in certain treatment regimens.

### BACKGROUND OF THE INVENTION

Substance use disorders (SUDs) affect almost 20 million adults and over 440,000 adolescents in the United States. Annually, it is estimated that more than 70,000 Americans die from drug overdoses, and that SUDs cost American society more than $740 billion annually in lost workplace productivity, healthcare expenses, and crime-related costs. Drug-related deaths in the United States have more than tripled since 2000, and there are more deaths, illness, and disabilities from substance use than from any other preventable health condition, to the extent that one in four deaths in the United States is currently attributable to alcohol, tobacco, and illicit or prescription drug use. To date there are only three FDA approved drugs for the treatment of alcohol use disorder—disulfiram, naltrexone, and acamprosate—and only three approved drugs for the treatment of Opioid Use Disorder—buprenorphine, methadone, and naltrexone—and none for many other SUDs, highlighting the need for new and effective treatments for SUDs.

### INCORPORATION BY REFERENCE

Each patent, publication, and non-patent literature cited in the application is hereby incorporated by reference in its entirety as if each was incorporated by reference individually. Unless specifically stated otherwise, reference to any document herein is not to be construed as an admission that the document referred to or any underlying information in the document is prior art in any jurisdiction, or forms part of the common general knowledge in the art.

### BRIEF SUMMARY OF THE INVENTION

The following presents a simplified summary of some embodiments of the invention in order to provide a basic understanding of the invention. This summary is not an extensive overview of the invention. It is not intended to identify key or critical elements of the invention or to delineate the scope of the invention. Its sole purpose is to present some embodiments of the invention in a simplified form as a prelude to the more detailed description that is presented later.

In one aspect, provided is a method of treating a substance use disorder in a subject in need thereof comprising: (a) administering to the subject a pharmaceutical composition comprising 5-MeO-DMT or a salt thereof; and (b) providing the subject with a therapy session between 1 week and 10 weeks after the administering of step (a). In some embodiments, the subject has reduced substance consumption prior to the administering of step (a). In some embodiments, the subject has reduced substance consumption for at least 1 day prior to the administering of step (a). In some embodiments, the subject has reduced substance consumption for at least 3, 4, 5, 6, or 7 days prior to step (a). In some embodiments, the subject has reduced substance consumption for 5-12 days. In some embodiments, the reduced substance consumption is abstaining from substance consumption. In some embodiments, the reduced substance consumption is abstaining from moderate to heavy substance consumption. In some embodiments, the administering of step (a) is performed sublingually, buccally, by intramuscular injection, or intravenous injection. In some embodiments, the administering of step (a) is performed by an intramuscular injection, or a plurality of intramuscular injections. In some embodiments, the administering of step (a) is performed by a plurality of intramuscular injections performed within a period of 1 to 8 hours. In some embodiments, the plurality of intramuscular injections is performed within a period of 2 to 4 hours. In some embodiments, the method further comprises repeating steps (a) and (b) after a treatment holiday. In some embodiments, the treatment holiday is between 2 weeks and 2 months.

In another aspect, provided is a method of treating a substance use disorder in a subject in need thereof comprising: (a) identifying the subject, wherein the subject has reduced substance consumption prior to the administration of step (b); and (b) administering to the subject a pharmaceutical composition comprising 5-MeO-DMT or a salt thereof. In some embodiments, the method further comprises providing the subject with a therapy session between 1 week and 10 weeks after the administering of step (b). In some embodiments, the subject has reduced substance consumption for at least 1 day prior to the administering of step (b). In some embodiments, the subject has reduced substance consumption for at least 3, 4, 5, 6, or 7 days prior to step (b). In some embodiments, the subject has reduced substance consumption for 5-12 days. In some embodiments, the reduced substance consumption is abstaining from substance consumption. In some embodiments, the reduced substance consumption is abstaining from moderate to heavy substance consumption. In some embodiments, the administering of step (b) is performed sublingually, buccally, by intramuscular injection, or intravenous injection. In some embodiments, the administering of step (b) is performed by an intramuscular injection, or a plurality of intramuscular injections. In some embodiments, the administering of step (b) is performed by a plurality of intramuscular injections performed within a period of 1 to 8 hours. In some embodiments, the plurality of intramuscular injections is performed within a period of 2 to 4 hours. In some embodiments, the method further comprises repeating steps (a) and (b) after a treatment holiday. In some embodiments, the treatment holiday is between 2 weeks and 2 months.

In some embodiments, the substance use disorder is alcohol use disorder; cannabis use disorder; hallucinogen use disorder; inhalant use disorder; opioid use disorder; sedatives, hypnotics, or anxiolytics use disorder; stimulant use disorder; tobacco or nicotine use disorder; or a combination thereof. In some embodiments, the substance use disorder is alcohol abuse disorder. In some embodiments, the substance use disorder is opioid abuse disorder. In some embodiments, the opioid use disorder comprises abuse of legal prescription opioids.

In some embodiments, the administering is performed by a licensed healthcare professional. In some embodiments, the therapy session comprises therapy with a licensed professional practitioner and at least one subject. In some embodiments, the therapy session comprises therapy with a licensed professional practitioner and a plurality of subjects. In some embodiments, the therapy session comprises psychosocial therapy.

In some embodiments, the 5-MeO-DMT is an HCl or sulfuric acid addition salt. In some embodiments, the 5-MeO-DMT is 5-MeO-DMT HCl. In some embodiments, the 5-MeO-DMT or salt thereof is not derived from a natural source. In some embodiments, the 5-MeO-DMT or salt thereof is produced by chemical synthesis. In some embodiments, the 5-MeO-DMT comprises less than 5% impurities. In embodiments, the 5-MeO-DMT comprises less than 1% of an impurity selected from animal cells, animal tissue, fungal cells, fungal tissue, plant cells, and plant tissue.

In some embodiments, the total dose of the 5-MeO-DMT or a salt thereof administered is between about 6 mg and 80 mg, 10 mg and 80 mg, 20 mg and 80 mg, or 25 mg and 80 mg. In some embodiments, the total dose of the 5-MeO-DMT or a salt thereof administered is between about 25 mg and 50 mg.

In some embodiments, treatment attenuates substance intake by the subject by about 20% or more, by about 30% or more, by about 40% or more, by about 50% or more, by about 60% or more, by about 70% or more, by about 80% or more, by about 90% or more, by about 95% or more, or up to about 100% as compared with the amount of substance intake before said treatment.

In some aspects are disclosed methods of treating a substance use disorder in a subject in need thereof comprising: (a) administering a first treatment to the subject, wherein the first treatment comprises administering by a plurality of intramuscular injections a first combined dose of 4 mg to 40 mg of 5-MeO-DMT or a salt thereof; and (b) administering a second treatment to the subject at least four weeks after the first treatment, wherein the second treatment comprises administering by a plurality of intramuscular injections a second combined dose of 5-MeO-DMT or a salt thereof, and wherein the second combined dose is equal to or greater than the first combined dose. In some embodiments, the method further comprises a first therapy session conducted between the first treatment and the second treatment and a second therapy session conducted after the second treatment. In some embodiments, the method further comprises one or more maintenance treatments comprising intramuscular injection of 5-MeO-DMT or a salt thereof to the subject and providing a maintenance therapy session after the treatment. In some embodiments, the first therapy session, the second therapy session, and the maintenance therapy session comprises a practitioner and a plurality of subjects. In some embodiments, the first therapy session, the second therapy session, and the maintenance therapy session each comprises the practitioner and at least one subject. In some embodiments, the first therapy session, the second therapy session, and the maintenance therapy session each comprises psychosocial therapy.

The foregoing outlines broadly some pertinent features of certain exemplary embodiments of this disclosure so the detailed description that follows may be better understood and so the present contribution to the art can be more fully appreciated. Additional features of the invention are described hereinafter which form the subject of the claims of the invention. It will be appreciated by those skilled in the art that the conception and the disclosed specific formulations and methods may be readily utilized as a basis for modifying or designing other formulations and methods for carrying out the same purposes of this disclosure. It will also be realized that such equivalent formulations and methods do not depart from the spirit and scope of the invention as set forth in the claims. Hence, this summary is made with the understanding that it will be considered as a brief and general synopsis of only some of the objects and embodiments herein, is provided solely for the benefit and convenience of the reader, and is not intended to limit in any manner the scope, or range of equivalents, to which the claims are lawfully entitled.

The scope of the invention includes all embodiments and formulations thereof, not only those expressly described below, and it will be understood that many modifications and variations in the described embodiments and details of the invention can be made by those skilled in the art without departing from the spirit of the invention, or the scope of the invention as set forth in the appended claims. Where methods described include particular steps, it will be apparent that other methods including fewer, more, or different steps than those described are also contemplated, and the description of steps in an exemplary order are not meant to limit the invention to that order.

The headings in this disclosure are utilized only to expedite its review by a reader. They should not be construed as limiting the invention, or any embodiments thereof, in any manner.

### DETAILED DESCRIPTION

This disclosure relates to methods of using 5-MeO-DMT to treat SUDs, such as alcohol use disorder, cannabis use disorder, hallucinogen use disorder, inhalant use disorder, opioid use disorder, sedatives, hypnotics, or anxiolytics use disorder, stimulant use disorder, tobacco or nicotine use disorder, or a combination thereof, such as a subject having more than one thereof.

In some aspects, the disclosure relates to methods of synthesizing 5-MeO-DMT, compositions containing 5-MeO-DMT, and methods of using 5-MeO-DMT, including its administration to subjects. In further aspects, the disclosure relates to 5-MeO-DMT administration and treatment regimens, which may include any of a reduction of a subject's substance intake prior to 5-MeO-DMT administration, provision of psychotherapy to a subject after 5-MeO-DMT administration, and a 5-MeO-DMT treatment holiday.

### A. General Definitions and Terms

Unless defined otherwise, all technical and scientific terms herein have the meaning as commonly understood by one having ordinary skill in the art to which this invention belongs, who as a shorthand may be referred to simply as “one of skill.” Further definitions that may assist the reader in understanding the disclosed embodiments follow; however, it will be appreciated that such definitions are for the purpose of describing particular embodiments only and are not intended to limit the scope of the invention, which shall be properly interpreted and understood by reference to the full specification, any plain meaning to one of skill, and the language of the claims.

As used in the specification and claims, the singular form “a”, “an” and “the” includes plural references unless the context clearly dictates otherwise. The terms “comprising,” “including,” “such as,” and “having” are intended to be inclusive and not exclusive (i.e., there may be other elements in addition to the recited elements). Thus, the term “including” as used herein means, and is used interchangeably with, the phrase “including but not limited to.”

The terms “subject,” “individual,” and “patient” are interchangeably and refer to humans, the as well as non-human mammals (e.g., primates, canines, equines, felines, porcines, bovines, ungulates, lagomorphs, and the like). In embodiments, the subject can be a human (e.g., adult male, adult female, adolescent male, adolescent female, male child, female child) under the care of a physician or other health worker in a hospital, as an outpatient, or other clinical context. In embodiments, the subject may not be under the care of a physician or other health worker.

The term “a subject in need thereof” refers to a subject that suffers from, or is at risk for, a pathology to be prophylactically or therapeutically treated with a disclosed compound or salt.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The term “substance use disorder” or “substance-related disorders” may refer to a diagnosis of disorders as defined under substance-related and addictive disorders by the Diagnostic and Statistical Manual of Mental Disorders (DSM-5). The term “substance use disorder” or “substance-related disorders” may include, inter alia, substance abuse and/or substance dependence. Non-limiting examples of substance use disorder include alcohol abuse disorder, stimulant abuse disorder, and opioid abuse disorder.

The term “risk drinking level” can be any of a “very high risk level,” “high risk level,” “moderate risk level,” or “low risk level.” In some embodiments (interchangeably as shorthand, “in embodiments”), the reduction in alcohol consumption is a reduction in the risk drinking level. In embodiments, the reduction in alcohol consumption is a reduction within the risk drinking level. In embodiments, the reduction in alcohol consumption is measured using the risk drinking level.

The term “high risk level” is defined as 60-100 g of alcohol for a male subject and 40-60 g of alcohol for a female subject. In some embodiments, “high risk level” is defined as 4.3-7.1 standard drinks for a male subject and 2.9-4.3 standard drinks for a female subject. The term “moderate risk level” is defined as 40-60 g of alcohol for a male subject and 20-40 g of alcohol for a female subject. In some embodiments, “moderate risk level” is defined as 2.9-4.3 standard drinks for a male subject and 1.4-2.9 standard drinks for a female subject. The term “low risk level” is defined as 1-40 g of alcohol for a male subject and 1-20 g of alcohol for a female subject. In some embodiments, “low risk level” is defined as less than 2.9 standard drinks for a male subject and less than 1.4 standard drinks for a female subject.

The term “binge drinking” may be defined using the standards provided by the National Institute on Alcohol Abuse and Alcoholism (NIAAA) or the World Health Organization (WHO). In embodiments, binge drinking is defined using the standards provided by NIAAA. In some embodiments, “binge drinking” is defined as a pattern of drinking alcohol that brings blood alcohol concentration (BAC) to 0.08 percent or higher. In embodiments, binge drinking is defined as a pattern of drinking alcohol that brings BAC to 0.08 grams of alcohol per deciliter or higher. For a typical adult, this pattern corresponds to consuming 5 or more drinks (male), or 4 or more drinks (female), in about 2 hours. In embodiments, a reduction in the pattern of drinking is a reduction in binge drinking. In embodiments, binge drinking is defined as using the standards proved by WHO. In embodiments, binge drinking is defined as heavy episodic drinking adults (aged>15 years) who consume at least 60 grams or more of pure alcohol at least once a week. In embodiments, a reduction in the consumption of pure alcohol once a week is a reduction in binge drinking.

The terms “administer”, “administered”, “administers” and “administering” are defined as providing a composition to a subject via a route known in the art, including intravenous, intraarterial, oral, parenteral, buccal, topical, transdermal, rectal, intramuscular, subcutaneous, intraosseous, transmucosal, or intraperitoneal routes of administration. In certain embodiments, oral routes of administering a composition can be used. The terms “administer”, “administered”, “administers” and “administering” a compound should be understood to mean providing a compound of the disclosure or a prodrug of a compound of the disclosure to the individual in need.

The term “plant tissue” refers to a part, or the whole, of an organism belonging to the Plantae kingdom. The term “plant cells” means a plurality of cells from an organism belonging to the Plantae kingdom. The term “animal tissue” refers to a part, or the whole, of an organism belonging to the Animalia kingdom. The term “animal cells” means a plurality of cells from an organism belonging to the Animalia kingdom. The term “fungal tissue” refers to a part, or the whole, of an organism belonging to the Fungi kingdom. The term “fungal cells” means a plurality of cells from an organism belonging to the Fungi kingdom.

“5-MeO-DMT” refers to 5-methoxy-N,N-dimethyltryptamine. 5-MeO-DMT is a naturally occurring tryptamine that agonizes 5-HTIA and 5-HT2A receptors. Non-linear pharmacokinetics of 5-MeO-DMT has been reported in rodents, indicating potentially unpredictable levels of subjective and physiological effects at higher doses. See, e.g., Shen et al., Drug Metab Dispos. 2011 Jul;39 (7): 1227-34 and Ermakova et al., J Psychopharmacol. 2022 Mar;36 (3): 273-294. Subjective effects of 5-MeO-DMT may include distorted perceptions of time, visual and auditory hallucinations, heightened emotional or transcendent states, and ego dissolution. The subjective experience of psychedelics, specifically the intensity of mystical experiences, has been correlated with positive treatment outcomes. See, e.g., Ko et al., Front Psychiatry. 2022 Jul 12; 13:917199. In addition to its subjective effects, the physiological effects of 5-MeO-DMT may be useful to treat aspects of substance addiction, including reduced neuroplasticity and inflammation. In one example, 5-MeO-DMT has been shown to significantly decrease IL-6 levels post-administration See, e.g., Uthaug et al., Psychopharmacol. (Berl). 2020 Mar;237 (3): 773-785.

The term “salt thereof” means a pharmaceutically acceptable salt, e.g., of 5-MeO-DMT.

The efficacy of 5-MeO-DMT or salt thereof; intramuscular, intravenous, or buccal formulation; or pharmaceutical compositions, as disclosed herein, may be measured as a score. The scores correspond to peak psychedelic experience, mystical experience, and the like. The occurrence of a peak psychedelic experience can be identified through achievement of at least 60% of the maximum possible score in each of the four subscales (mystical, positive mood, transcendence of time and space, and ineffability) of the 30-item revised Mystical Experience Questionnaire (MEQ30) as described in (Barrett F.S., J Psychopharmacol., 2015, 29, 1182-90, the entirety of which is incorporated by reference) or is identified through achievement of at least 60% of the maximum possible score of the Oceanic Boundlessness (OBN) dimension of the Altered States of Consciousness (ASC) questionnaire (R.L. et al., Front Pharmacol., 2018, 8, 974, the entirety of which is incorporated by reference) or through achievement of a Peak Psychedelic Experience Questionnaire (PPEQ) Total Score of at least 75.

In some embodiments, the efficacy of treatment, as disclosed herein, may be assessed using, for example, Food and Drug Administration (FDA) draft guidance as provided under “Alcoholism: Developing Drugs for Treatment” (February 2015). In some embodiments, the efficacy of the treatment, as disclosed herein, may be assessed using, for example, Food and Drug Administration (FDA) draft guidance as provided under “Opioid Use Disorder: Endpoints for Demonstrating Effectiveness of Drugs for Treatment Guidance for Industry” (October 2020).

The term “effective amount” or “therapeutically effective amount” refers to that amount of a disclosed compound or salt that is sufficient to affect the intended application including disease treatment, as defined below. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term can also apply to a dose that can induce a particular response in target cells, e.g., reduction of proliferation or down regulation of activity of a target protein. The specific dose can vary depending on the particular compounds chosen, the dosing regimen to be followed, whether it is administered in combination with other compounds, timing of administration, the tissue to which it is administered, and the physical delivery system in which it is carried.

The term “treatment” or “treating” refers to an approach for obtaining beneficial or desired results with respect to a disease, disorder, or medical condition including a therapeutic benefit and/or a prophylactic benefit. In embodiments, treatment or treating involves administering a disclosed compound or composition to a subject. A therapeutic benefit may include the eradication or amelioration of the underlying disorder being treated. Also, a therapeutic benefit may be achieved with the eradication or amelioration of one or more of the physiological symptoms associated with the underlying disorder, such as observing an improvement in the subject, notwithstanding that the subject may still be afflicted with the underlying disorder. In embodiments, for prophylactic benefit, a composition is administered to a subject at risk of developing a particular disease, or to a subject reporting one or more of the physiological symptoms of a disease, even though a diagnosis of this disease may not have been made. Treating can include, for example, reducing, delaying or alleviating the severity of one or more symptoms of the disease or condition, or it can include reducing the frequency with which symptoms of a disease, defect, disorder, or adverse condition, and the like, are experienced. Treating can refer to a method that results in some level of treatment or amelioration of the disease or condition, and can contemplate a range of results directed to that end, including prevention of the condition entirely.

The term “practitioner,” “medical practitioner,” and “medical professional” are defined as trained individuals in the medicinal arts and the like. For example, trained individuals can include psychiatrists, psychologists, addiction specialists, substance abuse counselors, or therapists.

The term “about” includes the recited number ±10%. Thus, “about 10” means 9 to 11.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, “greater than or equal to 1, 2, or 3” is equivalent to greater than or equal to 1, greater than or equal to 2, or greater than or equal to 3. Whenever the term “no more than,” “at most”, “less than,” or “less than or equal to” precedes the first numerical value in a series of two or more numerical values, the term “no more than,” “at most”, “less than,” or “less than or equal to” applies to each of the numerical values in that series of numerical values. For example, less than or equal to 3, 2, or 1 is equivalent to less than or equal to 3, less than or equal to 2, or less than or equal to 1. Every maximum numerical limitation includes every lower numerical limitation, as if such lower numerical limitations were expressly written herein. Every minimum numerical limitation will include every higher numerical limitation, as if such higher numerical limitations were expressly written herein. Every numerical range will include every narrower numerical range that falls within such broader numerical range, as if such narrower numerical ranges were all expressly written herein. The terms “decreased” or “decrease,” as used herein, generally mean a decrease by a statistically significant amount. In some embodiments, “decreased” or “decrease” means a reduction by at least 10% as compared to a reference level, for example, a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease from 10% to 100% as compared to a reference level. In the context of a marker or symptom, by these terms is meant a statistically significant decrease in such level. The decrease can be, for example, at least 10%, at least 20%, at least 30%, at least 40% or more.

### B. Methods of Treating Substance Use Disorders

In certain aspects, the present disclosure provides methods of treating SUDs using 5-MeO-DMT, for example, a pharmaceutical composition comprising 5-MeO-DMT or a salt thereof. In some aspects, provided is 5-MeO-DMT or a salt thereof for use in the treatment of substance use disorder (SUD). In further aspects, provided is use of 5-MeO-DMT or a salt thereof for the manufacture of a medicament for treating SUDs. Herein, “substance use disorder,” “substance use” and “substance dependence” may be used synonymously. In some embodiments, the provided methods comprise administering 5-MeO-DMT to a subject having an SUD. In some embodiments, the methods further comprise psychotherapy, such as psychedelic-assisted psychotherapy or 5-MeO-DMT-assisted psychotherapy.

In some embodiments, the subject reduces consumption of a substance prior to administration of a pharmaceutical composition comprising 5-MeO-DMT or a salt thereof. In some embodiments, the substance of reduced consumption is the substance of misuse or abuse. For example, if the subject has stimulant use disorder, specifically cocaine use disorder, the subject reduces consumption of cocaine prior to administration of 5-MeO-DMT. In some embodiments, the substance of reduced consumption may be more than one substance. For example, if the subject has stimulant use disorder, the subject may reduce consumption of all stimulants of abuse, e.g., caffeine, cocaine, and methamphetamine, prior to administration of a pharmaceutical composition comprising 5-MeO-DMT or a salt thereof. In some embodiments, the subject reduces consumption of psychoactive substances prior to administration of a pharmaceutical composition comprising 5-MeO-DMT or a salt thereof. In embodiments, the subject abstains from psychoactive substances prior to treatment with a pharmaceutical composition comprising 5-MeO-DMT or a salt thereof.

In some embodiments, the subject completes a detoxification regimen prior to treatment with 5-MeO-DMT. In some embodiments, the detoxification regimen comprises reducing consumption of a substance. In some embodiments, the detoxification regimen comprises reducing alcohol consumption. In some embodiments, the detoxification regimen comprises reducing cannabis consumption. In some embodiments, the detoxification regimen comprises reducing hallucinogen consumption. In some embodiments, the detoxification regimen comprises reducing inhalant consumption. In some embodiments, the detoxification regimen comprises reducing stimulant consumption. In some embodiments, the detoxification regimen comprises reducing tobacco or nicotine consumption. In some embodiments, the detoxification regimen comprises reducing opioid consumption. In some embodiments, the detoxification regimen comprises reducing sedative, hypnotic, anxiolytic consumption. In some embodiments, the detoxification regime is medically-assisted. Methods of determining reduced consumption are known to a skilled artisan and may include, e.g., biological specimen testing, self-reported measures of substance consumption, or a combination thereof.

In some embodiments, treating a subject having an SUD with 5-MeO-DMT results in a reduction of the subject's consumption of the substance. In some embodiments, the substance use disorder is any one or more of alcohol use disorder, cannabis use disorder, hallucinogen use disorder, inhalant use disorder, opioid use disorder, stimulant use disorder, sedative, hypnotic, and anxiolytic use disorder, or nicotine dependence and tobacco use disorder. In some embodiments, the substance is any of alcohol, caffeine, cannabis, a cannabinoid, a hallucinogen or psychedelic, an inhalant, an opioid or opiate, a stimulant, a sedative, hypnotic, or anxiolytic, or nicotine and/or tobacco. In some embodiments, the substance use disorder is selected from alcohol abuse disorder, stimulant abuse disorder, and opioid abuse disorder. For example, in some embodiments, the substance abuse disorder is alcohol abuse disorder. In other embodiments, the substance abuse disorder is opioid abuse disorder. In some embodiments, the opioid abuse disorder comprises abuse of legal prescription opioids. In some embodiments, the substance use disorder is selected from substance abuse and substance dependence.

Substance use disorders involve recurrent use of alcohol and/or drugs that causes clinically and functionally significant impairment, such as health problems, disability, and failure to meet major responsibilities at work, school, or home. According to the DSM-5, a diagnosis of substance use disorder is based on evidence of impaired control, social impairment, risky use, and pharmacological criteria. The DSM-5 establishes nine types of “substance-related” disorders: 1. Alcohol, 2. Caffeine, 3. Cannabis (e.g., marijuana), 4. Hallucinogens, 5. Inhalants, 6. Opioids (e.g., heroin), 7. Sedatives, Hypnotics, or Anxiolytics (e.g., benzodiazepines, barbiturates), 8. Stimulants (e.g., cocaine, methamphetamine), and 9. Tobacco. According to the DSM-5, each specific substance is addressed as a separate use disorder (i.e., alcohol use disorder, cannabis use disorder, hallucinogen use disorder, inhalant use disorder, opioid use disorder, sedative use disorder, stimulant use disorder, tobacco use disorder, and nicotine use disorder), but nearly all substances are diagnosed based on the same overarching criteria.

In some embodiments, a subject is identified as having a substance use disorder by a medical professional using symptoms or criteria as defined by DSM-5. In some embodiments, the subject is identified as having an SUD by a medical professional using symptoms or criteria as defined by ICD-11. In some embodiments, the subject is identified as having an SUD by a medical professional using symptoms or criteria as defined by a revision or new version of the DSM-5 or ICD-11, or by similar diagnostic criteria. In some embodiments, the substance use disorder in a subject is diagnosed by a medical professional. In some embodiments, the substance use disorder in a subject is diagnosed as a mild substance use disorder. In some embodiments, the substance use disorder in a subject is diagnosed as a moderate substance use disorder. In some embodiments, the substance use disorder in a subject is diagnosed as a severe substance use disorder.

In some examples, the symptoms of substance use disorders can include at least one of: 1. substance is often taken in larger amounts or over a longer period of time than was intended; 2. persistent desire or unsuccessful efforts to cut down or control substance use; 3. great deal of time is spent in activities necessary to obtain the substance, use the substance, or recover from its effects; 4. craving or strong desire to use the substance; 5. recurrent use resulting in failure to fulfill major role obligations at work, school, home; 6. continued substance use despite having persistent or recurrent social or interpersonal problems; 7. important social, occupational, or recreational activities are given up or reduced because of substance use; 8. recurrent substance use in situations in which it is physically hazardous; 9. substance use is continued despite knowledge of having a persistent or recurrent physical or psychological problem that is likely to have been caused or exacerbated by the substance; 10. tolerance, as defined by either of the following: a. a need for markedly increased amounts of the substance to achieve intoxication or desired effect, and b. a markedly diminished effect with continued use of the same amount of substance; and 11. withdrawal, as manifested by either of the following: a. characteristic withdrawal syndrome for the substance, or b. use of the substance or closely related substance is taken to relieve or avoid withdrawal symptoms, or a combination of any of the preceding symptoms.

a. Substance Use Disorders
i. Alcohol Use Disorder

In some aspects, provided are methods of using 5-MeO-DMT or a salt thereof to reduce a subject's alcohol intake. In some embodiments, the subject has alcohol use disorder (AUD). In some embodiments, 5-MeO-DMT or a salt thereof is used in the treatment of AUD. In some embodiments, a pharmaceutical composition comprising 5-MeO-DMT or a salt thereof is administered to a subject in need thereof to treat AUD. In some embodiments, the substance use disorder is an alcohol use disorder selected from alcohol abuse, alcohol dependence, and alcoholism. In some embodiments, the methods further comprise providing psychotherapy to the subject. In some embodiments, the subject reduces alcohol intake prior to treatment with 5-MeO-DMT. In some embodiments, “alcohol use disorder” (AUD) refers to the disorder as defined in the Diagnostic and Statistical Manual of Mental Disorders, for example, the DSM-5. Under DSM-5, anyone meeting any two of 11 listed criteria during the same 12 month period receive a diagnosis of AUD. The severity of AUD mild, moderate, or severe is based on the number of criteria met. In some embodiments, 5-MeO-DMT, either alone or in conjunction with psychotherapy, is useful to treat AUD.

Although herein the terms “alcohol use disorder” and “AUD” generally refer to the criteria in the DSM-5, or a patient with a diagnosis based thereon, it will be appreciated that the compositions and methods herein are equally applicable to subjects having the equivalent underlying disorder, whether that disorder is diagnosed based on the criteria in DSM-5 or in DSM-IV (which described two distinct disorders: alcohol abuse and alcohol dependence with specific criteria for each), whether the diagnosis is based on other clinically acceptable criteria (e.g., as “Alcohol Dependence Syndrome,” see Edwards, The Alcohol Dependence Syndrome: A Concept as Stimulus to Enquiry. Brit. J. Addiction, 81:171-183), or whether the patient has not yet had a formal clinical diagnosis.

In some embodiments, treating AUD comprises reducing any one or more of a subject's alcohol intake, a subject's alcohol cravings, and a subject's cue reactivity to substance stimuli. A reduction in alcohol use refers to reducing the amount or frequency of alcohol use, for example as assessed by urinalysis e.g., by measuring metabolites of alcohol in urine, such as ethyl glucuronide (EtG) or as assessed by using self reported alcohol use with standardized tools like the Timeline Follow Back self report. See, e.g., Robinson et al., Psychol Addict Behav., 2014;28 (1): 154-62; Sobell et al., Drug Alcohol Depend., 1996;42 (1): 49-5. Cravings, including reductions thereof, may be determined using a self-reported scale, e.g., the Penn Alcohol Craving Scale (Hartwell et al., Addict Behav Rep. 2019 Jun 18; 10:100198).

In some embodiments, reducing alcohol use or reduction of alcohol use refers to a reduction in drinks per day, a reduction in drinks per drinking day, or a reduction in the frequency of drinking, such as a reduction in the percentage of drinking days or percentage of heavy drinking days. In certain embodiments, a reduction in alcohol consumption is a reduction in the number of heavy drinking days. In some embodiments, reducing alcohol use or reduction of alcohol use refers to an increase in the time to drinking or the time to the first heavy drinking day. The term “drinking” drinks or “alcoholic drinks” as used herein, is understood in the context of “standard drinks such as spirits or blends that are intended for human consumption, wherein a “standard drink” equals 12 g ethanol. The term “heavy drinking day” refers to a day with a total alcohol consumption ≥60 g of ethanol for men and ≥40 g for women. In some embodiments, administration of 5-MeO-DMT to a subject results in reduced alcohol use. In some embodiments, the subject is an AUD patient.

ii. Cannabis Use Disorder

In some aspects, provided are methods of using 5-MeO-DMT or a salt thereof to reduce a subject's cannabis intake. In some embodiments, the subject has cannabis use disorder. In some embodiments, 5-MeO-DMT or a salt thereof is used in the treatment of cannabis use disorder. In some embodiments, a pharmaceutical composition comprising 5-MeO-DMT or a salt thereof is administered to a subject in need thereof to treat cannabis use disorder. In some embodiments, the subject reduces cannabis intake prior to treatment with 5-MeO-DMT. “Cannabis use disorder” (CUD) refers to the disorder as defined in the Diagnostic and Statistical Manual of Mental Disorders, for example, the DSM-5. In some embodiments, the methods further comprise providing psychotherapy to the subject.

In some embodiments, treating CUD comprises reducing any one or more of a subject's cannabis intake, a subject's cannabis cravings, and a subject's cue reactivity to substance stimuli. A reduction in cannabis use refers to reducing the amount or frequency of cannabis use, for example as assessed by biological specimen testing, e.g., urinalysis of delta-9-THC metabolites, such as 11-nor-delta-9-tetrahydrocannabinol-9-carboxylic acid (9-carboxy-THC), in urine, blood analysis, breath analysis, hair analysis, nail analysis, or as assessed by using a self-reported assessment, e.g., the Severity of Dependence Scale (SDS). See, e.g., DeGregorio et al., Sci Rep. 2021 Nov 23;11 (1): 22776), Curran et al., Psychol Med. 2019 Juy; 49 (9): 1574-1580, and Cobo-Golpe et al., J Anal Toxicol. 2021 Nov 9;45 (9): 969-975. In embodiments, CUD comprises use of one or more compounds or products containing such compound(s) that modulate activity of the cannabinoid 1 (CB1) receptor, such as the main psychoactive compound in cannabis, tetrahydrocannabinol (THC). In embodiments, CUD comprises use of cannabis, one or more natural or synthetic cannabinoids, or combinations thereof. Reductions in cravings and/or cue reactivity may be determined according to methods available to one of skill in the art, e.g., use of an assessment, such as the Marijuana Craving Questionnaire-17 (MCQ17). See also Norberg et al., Addiction. 2016 Nov; 111 (11): 1923-1934.

iii. Hallucinogen Use Disorder

In some aspects, provided are methods of using 5-MeO-DMT or a salt thereof to reduce a subject's hallucinogen intake. In some embodiments, the subject has hallucinogen use disorder. In some embodiments, 5-MeO-DMT or a salt thereof is used in the treatment of hallucinogen use disorder. In some embodiments, a pharmaceutical composition comprising 5-MeO-DMT or a salt thereof is administered to a subject in need thereof to treat hallucinogen use disorder. In some embodiments, the subject reduces hallucinogen intake prior to treatment with 5-MeO-DMT. “Hallucinogen use disorder” (HUD) refers to the disorder as defined in the Diagnostic and Statistical Manual of Mental Disorders, for example, the DSM-5. Herein, the words “hallucinogen” and “psychedelic” may be used interchangeably. In some embodiments, the methods further comprise providing psychotherapy to the subject.

In some embodiments, treating HUD comprises reducing any one or more of a subject's hallucinogen intake, a subject's hallucinogen cravings, and a subject's cue reactivity to substance stimuli. A reduction in hallucinogen use refers to reducing the amount or frequency of hallucinogen use, for example as assessed by biological specimen testing, e.g., blood analysis, breath analysis, hair analysis, nail analysis, or as assessed by using a self-reported assessment, e.g., the Severity of Dependence Scale (SDS). See, e.g., Jang et al., J Pharm Biomed Anal. 2015 Nov 10;115:138-43. Reductions in cravings and/or cue reactivity may be determined according to methods available to one of skill in the art, e.g., use of an assessment, such as the Brief Substance Craving Scale (BCBS).

iv. Inhalant Use Disorder

In some aspects, provided are methods of using 5-MeO-DMT or a salt thereof to reduce a subject's inhalant intake. In some embodiments, the subject has inhalant use disorder. In some embodiments, 5-MeO-DMT or a salt thereof is used in the treatment of inhalant use disorder. In some embodiments, a pharmaceutical composition comprising 5-MeO-DMT or a salt thereof is administered to a subject in need thereof to treat inhalant use disorder. In some embodiments, the subject reduces inhalant intake prior to treatment with 5-MeO-DMT. “Inhalant use disorder” (IUD) refers to the disorder as defined in the Diagnostic and Statistical Manual of Mental Disorders, for example, the DSM-5. In some embodiments, the methods further comprise providing psychotherapy to the subject.

In some embodiments, treating IUD comprises reducing any one or more of a subject's inhalant intake, a subject's inhalant cravings, and a subject's cue reactivity to substance stimuli. A reduction in inhalant use refers to reducing the amount or frequency of inhalant use, for example as assessed by biological specimen testing, e.g., blood analysis, such as detection of elevated liver enzymes, breath analysis, hair analysis, nail analysis, or as assessed by using a self-reported assessment, e.g., the Severity of Dependence Scale (SDS). See, e.g., Jain & Verma, J Pharm Bioallied Sci. 2016 Jan-Mar; 8 (1): 18-22. Reductions in cravings and/or cue reactivity may be determined according to methods available to one of skill in the art, e.g., use of an assessment, such as the Brief Substance Craving Scale (BCBS), Penn Alcohol Craving Scale—Inhalants (PACS-I), or the inhalant craving questionnaire (ICQ). See, e.g., Alonso-Matías et al., Adicciones. 2015 Dec 15;27 (4): 276-87, Jain et al., Asian J Psychiatr. 2017 Dec;30:202-207, and Kalaysiri et al., Psychiatry Res. 2018 Mar;261:61-67.

v. Opioid Use Disorder

In some aspects, provided are methods of using 5-MeO-DMT or a salt thereof to reduce a subject's opioid intake. In some embodiments, the subject has opioid use disorder. In some embodiments, 5-MeO-DMT or a salt thereof is used in the treatment of opioid use disorder. In some embodiments, a pharmaceutical composition comprising 5-MeO-DMT or a salt thereof is administered to a subject in need thereof to treat opioid use disorder. In some embodiments, the subject reduces opioid intake prior to treatment with 5-MeO-DMT. “Opioid use disorder” (OUD) refers to the disorder as defined in the Diagnostic and Statistical Manual of Mental Disorders, for example, the DSM-5. In some embodiments, the methods further comprise providing psychotherapy to the subject.

In some embodiments, treating OUD comprises reducing any one or more of a subject's opioid intake, a subject's opioid cravings, and a subject's cue reactivity to substance stimuli. A reduction in opioid use refers to reducing the amount or frequency of opioid use, for example as assessed by biological specimen testing, e.g., blood analysis, breath analysis, hair analysis, nail analysis, or as assessed by using an assessment, e.g., the Prescription Opioid Misuse and Abuse Questionnaire (POMAQ) and the Severity of Dependence Scale (SDS). See, e.g., Coyne et al., Curr Med Res Opin. 2021 Mar; 37 (3): 493-503, Kale, Am Fam Physician. 2019 Jan 1;99 (1):33-39, and Madry, Bioanalysis. 2016 May;8 (9): 953-64. Reductions in cravings and/or cue reactivity may be determined according to methods available to one of skill in the art, e.g., use of an assessment, such as the Brief Substance Craving Scale (BCBS), Visual Analog Scale (VAS), Desires for Drug Questionnaire (DDQ), Heroin Craving Questionnaire (HCQ), and Obsessive-Compulsive Drug Use Scale (OCDUS). See, e.g., Kleykamp et al., Drug Alcohol Depend. 2019 Dec 1;205:107639.

vi. Sedative, Hypnotic, or Anxiolytic Disorder

In some aspects, provided are methods of using 5-MeO-DMT or a salt thereof to reduce a subject's intake of a sedative, hypnotic, anxiolytic, or a combination thereof. In some embodiments, the subject has sedative, hypnotic, or anxiolytic use disorder. In some embodiments, 5-MeO-DMT or a salt thereof is used in the treatment of sedative, hypnotic, or anxiolytic disorder. In some embodiments, a pharmaceutical composition comprising 5-MeO-DMT or a salt thereof is administered to a subject in need thereof to treat sedative, hypnotic, anxiolytic use disorder. In some embodiments, the subject reduces sedative, hypnotic, or anxiolytic intake prior to treatment with 5-MeO-DMT. “Sedatives, hypnotics, or anxiolytics use disorder” (SHAUD) refers to the disorder as defined in the Diagnostic and Statistical Manual of Mental Disorders, for example, the DSM-5. In embodiments, the methods further comprise providing psychotherapy to the subject.

In some embodiments, treating SHAUD comprises reducing any one or more of a subject's sedative, hypnotic, or anxiolytic intake, a subject's sedative, hypnotic, or anxiolytic cravings, and a subject's cue reactivity to substance stimuli. A reduction in sedative, hypnotic, or anxiolytic use refers to reducing the amount or frequency of sedative, hypnotic, or anxiolytic use, for example as assessed by biological specimen testing, e.g., blood analysis, breath analysis, hair analysis, nail analysis, or as assessed by using an assessment, e.g., the Severity of Dependence Scale (SDS). See, e.g., Tenore, Addict Dis. 2010 Oct;29 (4): 436-48. Reductions in cravings and/or cue reactivity may be determined according to methods available to one of skill in the art, e.g., use of an assessment, such as the Brief Substance Craving Scale (BCBS) or the Benzodiazepine Craving Questionnaire. See, e.g., Mol et al., Addiction. 2003 Aug;98 (8): 1143-52.

vii. Stimulant Use Disorder

In some aspects, provided are methods of using 5-MeO-DMT to reduce a subject's stimulant intake. In some embodiments, the subject has stimulant use disorder. In some embodiments, the subject has caffeine use disorder. In some embodiments, 5-MeO-DMT or a salt thereof is used in the treatment of stimulant use disorder. In some embodiments, a pharmaceutical composition comprising 5-MeO-DMT or a salt thereof is administered to a subject in need thereof to treat stimulant use disorder. In some embodiments, the subject reduces stimulant intake prior to treatment with 5-MeO-DMT. “Stimulant use disorder” (STUD) refers to the disorder as defined in the Diagnostic and Statistical Manual of Mental Disorders, for example, the DSM-5. In some embodiments, the methods further comprise providing psychotherapy to the subject.

In some embodiments, treating STUD comprises reducing any one or more of a subject's stimulant intake, a subject's stimulant cravings, and a subject's cue reactivity to substance stimuli. A reduction in stimulant use refers to reducing the amount or frequency of stimulant use, for example as assessed by biological specimen testing, e.g., blood analysis, breath analysis, hair analysis, nail analysis, or as assessed by using a self-reported assessment, e.g., the Severity of Dependence Scale (SDS). Reductions in cravings and/or cue reactivity may be determined according to methods available to one of skill in the art, e.g., use of an assessment, such as the Brief Substance Craving Scale (BCBS) or the Stimulant Craving Questionnaire-Brief (STCQ).

viii. Nicotine or Tobacco Use Disorder

In some aspects, provided are methods of using 5-MeO-DMT to reduce a subject's nicotine or tobacco intake. In some embodiments, the subject has nicotine or tobacco use disorder. In some embodiments, 5-MeO-DMT or a salt thereof is used in the treatment of nicotine or tobacco use disorder. In some embodiments, a pharmaceutical composition comprising 5-MeO-DMT or a salt thereof is administered to a subject in need thereof to treat nicotine or tobacco use disorder. In some embodiments, the subject reduces nicotine and/or tobacco intake prior to treatment with 5-MeO-DMT. “Nicotine or tobacco use disorder” (NTUD) refers to the disorder as defined in the Diagnostic and Statistical Manual of Mental Disorders, for example, the DSM-5. In some embodiments, the methods further comprise providing psychotherapy to the subject.

In some embodiments, treating NTUD comprises reducing any one or more of a subject's nicotine and/or tobacco intake, a subject's nicotine and/or tobacco cravings, and a subject's cue reactivity to substance stimuli. A reduction in nicotine and/or tobacco use refers to reducing the amount or frequency of nicotine and/or tobacco use, for example as assessed by biological specimen testing, e.g., blood analysis, breath analysis, hair analysis, nail analysis, or as assessed by using a self-reported assessment, e.g., the Severity of Dependence Scale (SDS) or the Fagerström Test for Nicotine Dependence. See, e.g., Rustin, Am Fam Physician. 2000 Aug 1;62 (3): 579-84, 591-2. Reductions in cravings and/or cue reactivity may be determined according to methods available to one of skill in the art, e.g., use of an assessment, such as the Brief Substance Craving Scale (BCBS) or the Questionnaire of Smoking Urges (QSU).

b. Treating Substance Use Disorder Based on Reduced Substance Consumption

In some aspects, provided herein are methods of using 5-MeO-DMT or a salt thereof to treat a subject having an SUD, wherein the subject reduces consumption of the substance prior to administration of 5-MeO-DMT. In some embodiments, the method for treating an SUD comprises identifying a subject for therapy (“identifying step”), wherein the subject has reduced substance consumption for at least 1 day prior to the administering of 5-MeO-DMT or a salt thereof. In some embodiments, the method for treating an SUD comprises administering to the subject a pharmaceutical composition comprising 5-MeO-DMT or a salt thereof, wherein the administering is performed by intramuscular injection, intravenous injection, or buccal administration. In some embodiments, the method for treating an SUD comprises providing the subject with a therapy session between 1 and 10 weeks following the administering of 5-MeO-DMT or a salt thereof.

In some embodiments, the identifying step comprises screening a subject before administration of 5-MeO-DMT or a salt thereof. In some embodiments, the screening comprises selecting a subject that has naive psychedelic exposure or limited psychedelic exposure. In another embodiment, the naive psychedelic exposure means that a subject had not ever consumed a psychedelic substance prior to the identifying step. In another embodiment, the limited psychedelic exposure means that a subject has consumed psychedelic substances less than 10 times in their lifetime, prior to the identifying step.

In some embodiments, the screening further comprises a reviewing step. In some embodiments, the screening comprises reviewing family psychiatric history for a relevant psychiatric disorder in first-degree relatives or second-degree relatives. In some embodiments, a subject may be excluded from administration of 5-MeO-DMT if the subject's first-degree relative has a psychiatric disorder. In some embodiments, a subject may be excluded from administration of 5-MeO-DMT if the subject's second-degree relative has a psychiatric disorder. In some embodiments, the screening comprises reviewing the subject's previous use of psychedelics. In some embodiments, the psychedelics are selected from psilocybin, LSD, DMT, ayahuasca, mescaline, ibogaine, 2C-drugs (such as 2CB, 2CI and 2CE), THC, and/or ketamine. In some embodiments, the screening comprises reviewing the subject's previous use of psychedelics selected from psilocybin, LSD, DMT, ayahuasca, mescaline, ibogaine, 2CB, 2CI, 2CE, THC, and ketamine. In some embodiments, a subject may be excluded from administration of 5-MeO-DMT if they had previous use of psychedelics. In some embodiments, the screening comprises reviewing the subject for a psychiatric adverse effect associated from the previous use of psychedelics. In some embodiments, the psychiatric adverse effect is selected from anxiety, sense of loss of control, paranoid ideation, paranoid behavior, hallucinatory behavior, euphoria, and suicidal ideation. In some embodiments, the screening comprises reviewing the subject for a psychiatric adverse effect selected from anxiety, sense of loss of control, paranoid ideation, paranoid behavior, hallucinatory behavior, euphoria, and suicidal ideation associated with the previous use of psychedelics. In some embodiments, a subject may be excluded from administration of 5-MeO-DMT if they had psychiatric adverse effects associated with the previous use of psychedelics. In some embodiments, the screening comprises reviewing the subject's persistent psychological effects. In some embodiments, the persistent psychological effects are selected from anxiety, depressed mood, paranoid ideation, hallucinations, flash-backs and recurrent flash-backs. In some embodiments, the screening comprises reviewing the subject for a history of suicidal ideation, suicidal behavior, or a history of suicidal attempts. In some embodiments, a subject may be excluded from administration of 5-MeO-DMT if they had persistent psychological effects. In some embodiments, a subject may be excluded from administration of 5-MeO-DMT if they have a history of suicidal ideation, suicidal behavior, or a history of suicidal attempts. In some embodiments, the screening comprises reviewing the subject for Hallucinogen Persisting Perception Disorder (HPPD). In some embodiments, a subject may be excluded from administration of 5-MeO-DMT if they have HPPD. In embodiments, the method further comprises obtaining informed written consent from the subject prior to administration of 5-MeO-DMT.

In some embodiments, the reduced substance consumption is abstaining from moderate to heavy substance consumption. In some embodiments, the reduced substance consumption is measured by a reduction in heavy use days. In some embodiments, the reduced substance consumption is measured by reduction in risk of use level. In some embodiments, the reduced substance consumption is abstaining from substance consumption. In some embodiments, the reduced substance consumption is a reduction in use. In some embodiments, the reduced substance consumption is abstaining from use.

In some embodiments, administering said treatment attenuates substance intake by said subject by about 20% or more as compared with the amount of substance intake before said treatment. In some embodiments, the administering said treatment attenuates substance intake by said subject by about 30%, about 40%, about 50%, about 60%, about 70%, about 80%, about 90%, or about 95% or more as compared with the amount of substance intake before said treatment. In some embodiments, the administering said treatment attenuates substance intake by said subject by at least 20%, at least 30%, at least 40%, at least 50%, at least 60%, at least 70%, at least 80%, at least 90%, or at least 95% as compared with the amount of substance intake before said treatment.

In some embodiments, the substance is any of alcohol, cannabis, a cannabinoid, a hallucinogen or psychedelic, an inhalant, an opioid or opiate, a stimulant, a sedative, hypnotic, or anxiolytic, or nicotine and/or tobacco. In some embodiments, the SUD is selected from substance abuse and substance dependence. In some embodiments, the SUD is substance abuse. In some embodiments, the SUD is substance dependence. In some embodiments, the SUD is a substance abuse disorder as defined under DSM-5.

In some embodiments, the SUD is selected from alcohol abuse disorder, cannabis use disorder, hallucinogen use disorder, inhalant use disorder, opioid use disorder, sedatives, hypnotics, or anxiolytics use disorder, stimulant use disorder, and tobacco or nicotine use disorder, or a combination thereof. In some embodiments, the substance use disorder is alcohol use disorder. In some embodiments, the substance use disorder is cannabis use disorder. In some embodiments, the substance use disorder is inhalant use disorder. In some embodiments, the substance use disorder is opioid use disorder. In some embodiments, the substance use disorder is sedatives, hypnotics, or anxiolytics use disorder. In some embodiments, the substance use disorder is stimulant use disorder. In some embodiments, the substance use disorder is tobacco or nicotine use disorder.

In some embodiments, the SUD is alcohol use disorder. In some such embodiments, the reduced substance consumption is measured by a reduction in heavy drinking days. In some embodiments, the reduced substance consumption is measured by reduction in risk drinking level. In some embodiments, the reduced substance consumption is abstaining from substance consumption. In some embodiments, the reduced substance consumption is a reduction in binge drinking as defined herein. In some embodiments, the reduced substance consumption is abstaining from binge drinking.

In some embodiments, the SUD is cannabinoid use disorder. In some such embodiments, the reduced substance consumption is measured by a reduction in heavy cannabinoid use days. In some embodiments, the reduced substance consumption is abstaining from cannabinoid consumption.

In some embodiments, the SUD is hallucinogen use disorder. In some such embodiments, the reduced substance consumption is measured by a reduction in heavy hallucinogen use days. In some embodiments, the reduced substance consumption is abstaining from hallucinogen consumption.

In some embodiments, the SUD is inhalant use disorder. In some such embodiments, the reduced substance consumption is measured by a reduction in heavy inhalant use days. In some embodiments, the reduced substance consumption is abstaining from inhalant consumption.

In some embodiments, the SUD is opioid use disorder. In some embodiment, the opioid abuse disorder comprises use and/or abuse of legal opioids, including, for example, legal prescription opioids. Such legal prescription opioids include, for example, hydrocodone, oxycodone, oxymorphone, morphine, codeine, hydromorphone, tapentadol, fentanyl, methadone, and the like. In some embodiments the opioid abuse disorder comprises use and/or abuse of illegal opioids, including, for example, heroin and/or fentanyl. In some such embodiments, the reduced substance consumption is measured by a reduction in heavy opioid use days. In embodiments, the reduced substance consumption is abstaining from opioid consumption.

In some embodiments, the SUD is sedative, hypnotic, or anxiolytic use disorder. In some such embodiments, the reduced substance consumption is measured by a reduction in heavy sedative, hypnotic, or anxiolytic use days. In some embodiments, the reduced substance consumption is abstaining from sedative, hypnotic, or anxiolytic consumption.

In some embodiments, the SUD is stimulant use disorder. In some such embodiments, the reduced substance consumption is measured by a reduction in heavy stimulant use days. In some embodiments, the reduced substance consumption is abstaining from stimulant consumption.

In some embodiments, the SUD is tobacco or nicotine use disorder. In some such embodiments, the reduced substance consumption is measured by a reduction in heavy tobacco or nicotine use days. In some embodiments, the reduced substance consumption is abstaining from tobacco or nicotine consumption.

In some embodiments, for the identifying step, said subject has reduced substance consumption for at least 1, 2, 3, 4, 5, 6, or 7 days prior to administration of 5-MeO-DMT or a salt thereof. In some embodiments, for the identifying step, said subject has reduced substance consumption for at least 3, 4, 5, 6, or 7 days prior to administration of 5-MeO-DMT or a salt thereof. In some embodiments, for the identifying step, said subject has reduced substance consumption for more than 1, 2, 3, 4, 5, 6, or 7 days prior to administration of 5-MeO-DMT or a salt thereof. In some embodiments, for the identifying step, said subject has reduced substance consumption for more than 3, 4, 5, 6, or 7 days prior to administration of 5-MeO-DMT or a salt thereof. In some embodiments, for the identifying step, said subject has reduced substance consumption for at least 2 days prior to administration of 5-MeO-DMT or a salt thereof. In some embodiments, for the identifying step, said subject has reduced substance consumption for at least 3 days prior to administration of 5-MeO-DMT or a salt thereof. In some embodiments, for the identifying step, said subject has reduced substance consumption for at least 4 days prior to administration of 5-MeO-DMT or a salt thereof. In some embodiments, for the identifying step, said subject has reduced substance consumption for at least 5 days prior to administration of 5-MeO-DMT or a salt thereof. In some embodiments, for the identifying step, said subject has reduced substance consumption for at least 6 days prior to administration of 5-MeO-DMT or a salt thereof. In some embodiments, for the identifying step, said subject has reduced substance consumption for at least 7 days prior to administration of 5-MeO-DMT or a salt thereof. In some embodiments, for the identifying step, said subject has reduced substance consumption for more than 2 days prior to administration of 5-MeO-DMT or a salt thereof. In some embodiments, for the identifying step, said subject has reduced substance consumption for more than 3 days prior to administration of 5-MeO-DMT or a salt thereof. In some embodiments, for the identifying step, said subject has reduced substance consumption for more than 4 days prior to administration of 5-MeO-DMT or a salt thereof. In some embodiments, for the identifying step, said subject has reduced substance consumption for more than 5 days prior to administration of 5-MeO-DMT or a salt thereof. In some embodiments, for the identifying step, said subject has reduced substance consumption for more than 6 days prior to administration of 5-MeO-DMT or a salt thereof. In some embodiments, for the identifying step, said subject has reduced substance consumption for more than 7 days prior to administration of 5-MeO-DMT or a salt thereof. In some embodiments, for the identifying step, said subject has reduced substance consumption for more than 1 week prior to administration of 5-MeO-DMT or a salt thereof. In some embodiments, for the identifying step, said subject has reduced substance consumption for more than 2 weeks prior to administration of 5-MeO-DMT or a salt thereof.

In some embodiments, the subject has reduced substance consumption for 1-24 days. In some embodiments, the subject has reduced substance consumption for 2-24 days. In some embodiments, the subject has reduced substance consumption for 3-18 days. In some embodiments, the subject has reduced substance consumption for 4-18 days. In some embodiments, the subject has reduced substance consumption for 5-12 days. In some embodiments, the subject has reduced substance consumption for 5-11 days. In some embodiments, the subject has reduced substance consumption for 5-10 days. In some embodiments, the subject has reduced substance consumption for 5-9 days. In some embodiments, the subject has reduced substance consumption for 5-8 days. In some embodiments, the subject has reduced substance consumption for 5-7 days. In some embodiments, the subject has reduced substance consumption for 5-6 days. In some embodiments, the subject has reduced substance consumption for 6-12 days. In some embodiments, the subject has reduced substance consumption for 7-12 days. In some embodiments, the subject has reduced substance consumption for 8-12 days. In some embodiments, the subject has reduced substance consumption for 9-12 days. In some embodiments, the subject has reduced substance consumption for 10-12 days. In some embodiments, the subject has reduced substance consumption for 11-12 days.

In some embodiments, the subject completes a detoxification regimen prior to treatment with 5-MeO-DMT or a salt thereof. In some embodiments, the detoxification regimen comprises reducing consumption of a substance. In some embodiments, the detoxification regimen comprises reducing alcohol consumption. In some embodiments, the detoxification regimen comprises reducing cannabis consumption. In some embodiments, the detoxification regimen comprises reducing hallucinogen consumption. In some embodiments, the detoxification regimen comprises reducing inhalant consumption. In some embodiments, the detoxification regimen comprises reducing stimulant consumption. In some embodiments, the detoxification regimen comprises reducing opioid consumption. In some embodiments, the detoxification regimen comprises reducing sedative, hypnotic, anxiolytic consumption. In some embodiments, the detoxification regimen comprises reducing tobacco or nicotine consumption. In some embodiments, the detoxification regime is medically-assisted. Methods of determining reduced consumption are known to a skilled artisan and may include, e.g., biological specimen testing, self-reported measures of substance consumption, or a combination thereof.

In some embodiments, further comprising a 5-MeO-DMT treatment holiday of from 2 weeks to 12 months. In some embodiments, the 5-MeO-DMT treatment holiday is from 2 weeks to 11 months. In some embodiments, the 5-MeO-DMT treatment holiday is from 2 weeks to 10 months. In some embodiments, the 5-MeO-DMT treatment holiday is from 2 weeks to 9 months. In some embodiments, the 5-MeO-DMT treatment holiday is from 2 weeks to 8 months. In some embodiments, the 5-MeO-DMT treatment holiday is from 2 weeks to 7 months. In some embodiments, the 5-MeO-DMT treatment holiday is from 2 weeks to 6 months. In some embodiments, the 5-MeO-DMT treatment holiday is from 2 weeks to 5 months. In some embodiments, the 5-MeO-DMT treatment holiday is from 2 weeks to 4 months. In some embodiments, the 5-MeO-DMT treatment holiday is from 2 weeks to 3 months. In some embodiments, the 5-MeO-DMT treatment holiday is from 2 weeks to 2 months. In some embodiments, the 5-MeO-DMT treatment holiday is from 2 weeks to 1 month. In some embodiments, the 5-MeO-DMT treatment holiday is from 2 weeks to 4 weeks. In some embodiments, the 5-MeO-DMT treatment holiday is from 2 weeks to 3 weeks. In some embodiments, the 5-MeO-DMT treatment holiday is from 1 month, 2 months, 3 months, 4, month, 5 months, 6 months, 7 months, 8 months, 9 months, 10 months, 11 months, or 12 months. In some embodiments, the 5-MeO-DMT treatment holiday is from 2 weeks, 3 weeks, 4 weeks, 5 weeks, 6 weeks, 7 weeks, 8 weeks. 10 weeks, 12 weeks, 14 weeks, 18 weeks, 20 weeks, 22 weeks, 24 weeks, 28 weeks, 32 weeks, 36 weeks, 40 weeks, 44 weeks, 48 weeks, or 52 weeks.

In some embodiments, the pharmaceutical composition comprises an acid addition salt of 5-MeO-DMT. In some embodiments, the acid addition salt of 5-MeO-DMT is selected from the HCl addition salt, the sulfuric acid addition salt, and the succinic acid addition salt. In some embodiments, the pharmaceutical composition comprises the HCl addition salt of 5-MeO-DMT. In some embodiments, the pharmaceutical composition comprises the sulfuric acid addition salt of 5-MeO-DMT. In some embodiments, the pharmaceutical composition comprises the succinic acid addition salt of 5-MeO-DMT.

In some embodiments, the administration of a pharmaceutical composition comprising 5-MeO-DMT or a salt thereof is performed by intramuscular injection, intravenous injection, or buccal administration. In some embodiments, the administration of a pharmaceutical composition comprising 5-MeO-DMT or a salt thereof is performed by intramuscular injection. Intramuscular administration can avoid the rapid onset and short duration of some other routes of administration. In some embodiments, the administration of a pharmaceutical composition comprising 5-MeO-DMT or a salt thereof is performed by intravenous injection. In some embodiments, the administration of a pharmaceutical composition comprising 5-MeO-DMT or a salt thereof is performed by buccal administration.

In some embodiments, the administration of 5-MeO-DMT or salt thereof is performed by a medical professional. In some embodiments, the administration of 5-MeO-DMT or salt thereof is performed by a licensed healthcare professional. In some embodiments, the administration of 5-MeO-DMT or salt thereof is performed by a licensed medical professional.

In some embodiments, the administration of 5-MeO-DMT or salt thereof is performed by a plurality of intramuscular injections. In some embodiments, the administration of 5-MeO-DMT or salt thereof is performed by a plurality of intramuscular injections performed within a period of 1 to 8 hours. In some embodiments, the administration of 5-MeO-DMT or salt thereof is performed by a plurality of intramuscular injections performed within a period of, for example, 1 to 10 hours, 1 to 9 hours, 1 to 8 hours, 1 to 7 hours, 1 to 6 hours, 1 to 5 hours, 1 to 4 hours, 1 to 3 hours, or 1 to 2 hours. In some embodiments, the administrating of 5-MeO-DMT or a salt thereof is performed by a plurality of intramuscular injections performed within a period of, for example, 2 to 10 hours, 2 to 9 hours, 2 to 8 hours, 2 to 7 hours, 2 to 6 hours, 2 to 5 hours, 2 to 4 hours, or 2 to 3 hours. In some embodiments, each of the plurality of injection is spaced apart by between 30 minutes and 2 hours. In some embodiments, each of the plurality of injection is spaced apart by between 30 minutes and 1.5 hours. In some embodiments, each of the plurality of injection is spaced apart by between 30 minutes and 1 hour. In some embodiments, each of the plurality of injection is spaced apart by between about 30 minutes, about 1 hour, about 1.5 hours, or about 2 hours

In some embodiments, the total dose of 5-MeO-DMT or salt thereof administered to a subject is from about 4 mg to about 60 mg, about 4 mg to about 50 mg, about 4 mg to about 40 mg, about 4 mg to about 30 mg, about 4 mg to about 20 mg, or about 4 mg to about 10 mg. In some embodiments, the total dose of 5-MeO-DMT or salt thereof administered to a subject is from about 8 mg to about 60 mg, about 8 mg to about 50 mg, about 8 mg to about 40 mg, about8 mg to about 30 mg, about 8 mg to about 20 mg, or about 8 mg to about 16 mg. In some embodiments, the total dose of 5-MeO-DMT or salt thereof administered to a subject is from about 12 mg to about 60 mg, about 12 mg to about 50 mg, about 12 mg to about 40 mg, or about 12 mg to about 30 mg. In some embodiments, the total dose of 5-MeO-DMT or salt thereof administered to a subject is from about 25 mg to about 80 mg, about 25 mg to 75 mg, about 25 mg to 70 mg, about 25 mg to 65 mg, about 25 mg to 60 mg, about 25 mg to 55 mg, about 25 mg to about 50 mg, about 25 mg to about 45 mg, or about 25 mg to about 30 mg. In some embodiments, the total dose of 5-MeO-DMT or salt thereof administered to a subject is from about 35 mg to about 80 mg, about 35 mg to about 75 mg, about 35 mg to about 70 mg, about 35 mg to about 65 mg, about 35 mg to about 60 mg, or about 25 mg to about 55 mg, about 35 mg to about 50 mg, about 35 mg to about 45 mg, or about 35 mg to about 40 mg.

In some embodiments, the pharmaceutical composition administered to a subject comprises from about 4 mg to about 60 mg of 5-MeO-DMT or salt thereof. In some embodiments, the pharmaceutical composition administered to a subject comprises from about 4 mg to about 50 mg of 5-MeO-DMT or salt thereof. In some embodiments, the pharmaceutical composition administered to a subject comprises from about 4 mg to about 40 mg of 5-MeO-DMT or salt thereof. In some embodiments, the pharmaceutical composition administered to a subject comprises from about 4 mg to about 30 mg of 5-MeO-DMT or salt thereof. In some embodiments, the pharmaceutical composition administered to a subject comprises from about 4 mg to about 20 mg of 5-MeO-DMT or salt thereof. In some embodiments, the pharmaceutical composition administered to a subject comprises from about 4 mg to about 10 mg of 5-MeO-DMT or salt thereof. In some embodiments, the pharmaceutical composition administered to a subject comprises from about 8 mg to about 60 mg of 5-MeO-DMT or salt thereof. In some embodiments, the pharmaceutical composition administered to a subject comprises from about 8 mg to about 50 mg of 5-MeO-DMT or salt thereof. In some embodiments, the pharmaceutical composition administered to a subject comprises from about 8 mg to about 40 mg of 5-MeO-DMT or salt thereof. In some embodiments, the pharmaceutical composition administered to a subject comprises from about 8 mg to about 30 mg of 5-MeO-DMT or salt thereof. In some embodiments, the pharmaceutical composition administered to a subject comprises from about 8 mg to about 20 mg of 5-MeO-DMT or salt thereof. In some embodiments, the pharmaceutical composition administered to a subject comprises from about 8 mg to about 16 mg of 5-MeO-DMT or salt thereof. In some embodiments, the pharmaceutical composition administered to a subject comprises from about 12 mg to about 60 mg of 5-MeO-DMT or salt thereof. In some embodiments, the pharmaceutical composition administered to a subject comprises from about 12 mg to about 50 mg of 5-MeO-DMT or salt thereof. In some embodiments, the pharmaceutical composition administered to a subject comprises from about 12 mg to about 40 mg of 5-MeO-DMT or salt thereof.

In some embodiments, the pharmaceutical composition administered to a subject comprises at least 5 mg, at least 10 mg, at least 15 mg, at least 20 mg, at least 25 mg, at least 30 mg, or at least 40 mg of 5-MeO-DMT or a salt thereof. In some embodiments, the pharmaceutical composition administered to a subject comprises at most 80 mg, at most 70 mg, at most 60 mg, at most 50 mg, at most 45 mg, at most 40 mg, or at most 35 mg of 5-MeO-DMT or a salt thereof.

In some embodiments, the therapy session comprises psychosocial therapy. In some embodiments, the therapy session comprises a practitioner and at least one subject. In some embodiments, the therapy session is a group session with the plurality of subjects. In some embodiments, the group session comprises the practitioner and at least two subjects. In some embodiments, the therapy session comprises a practitioner and a plurality of subjects. In some embodiments, the practitioner is a trained medical professional. For example, the trained medical professional can include but is not limited to psychiatrists, psychologists, addiction specialists, substance abuse counselors, and therapists. In some embodiments, the trained medical profession is a psychiatrist, a psychologist, an addiction specialist, a substance abuse counselor, a therapist, or any combination thereof. In some embodiments, the therapy session comprises the practitioner and at least one subject.

In some embodiments, the therapy session occurs between 1 week and 10 weeks after the administration of the 5-MeO-DMT or salt thereof. In some embodiments, the therapy session occurs between 2 weeks and 10 weeks after the administration of the 5-MeO-DMT or salt thereof. In some embodiments, the therapy session occurs between 3 weeks and 10 weeks after the administration of the 5-MeO-DMT or salt thereof. In some embodiments, the therapy session occurs between 4 weeks and 10 weeks after the administration of the 5-MeO-DMT or salt thereof. In some embodiments, the therapy session occurs between 1 week and 8 weeks after the administration of the 5-MeO-DMT or salt thereof. In some embodiments, the therapy session occurs between 2 weeks and 8 weeks after the administration of the 5-MeO-DMT or salt thereof. In some embodiments, the therapy session occurs between 3 weeks and 8 weeks after the administration of the 5-MeO-DMT or salt thereof. In some embodiments, the therapy session occurs between 4 weeks and 8 weeks after the administration of the 5-MeO-DMT or salt thereof. In some embodiments, the therapy session occurs at least 1 day after administration of the 5-MeO-DMT or salt thereof. In some embodiments, the therapy session occurs at least, for example, 2 days, 3 days, 4 days, 5 days, 6 days, 7 days, 8 days, 9 days, or 10 days after administration of the 5-MeO-DMT or salt thereof. In some embodiments, the therapy session occurs at least, for example, 1 week, 2 weeks, 3 weeks, 4 weeks, 5 weeks, or 6 weeks after administration of the 5-MeO-DMT or salt thereof.

In some embodiments, the pharmaceutical composition comprises less than 5% impurities. In some embodiments, the pharmaceutical composition comprises less than 5% impurities, less than 4% impurities, less than 3% impurities, less than 2% impurities, less than 1% impurities, less than 0.5% impurities, less than 0.4% impurities, less than 0.3% impurities, less than 0.2% impurities, or less than 0.1% impurities. In some embodiments, the pharmaceutical composition comprises at most 5% impurities, at most 4% impurities, at most 3% impurities, at most 2% impurities, at most 1% impurities, at most 0.5% impurities, at most 0.4% impurities, at most 0.3% impurities, at most 0.2% impurities, or at most 0.1% impurities.

In some embodiments, the impurity is selected from animal cells, animal tissue, fungal cells, fungal tissue, plant cells, and plant tissue. In some embodiments, the impurity is derived from animal cells, animal tissue, fungal cells, fungal tissue, plant cells, or plant tissue. In some embodiments, the impurity is selected from animal cells. In some embodiments, the impurity is selected from animal tissue. In some embodiments, the animal cells or animal tissue are derived from Bufo alvarius. In some embodiments, the impurity is selected from fungal cells. In some embodiments, the impurity is selected from fungal tissue. In some embodiments, the fungal cells or fungal tissue are derived from Amanita citrina, Amanita porphyria, or a combination thereof. In some embodiments, the impurity is selected from plant cells. In some embodiments, the impurity is selected from plant tissue. In some embodiments, the plant cells or plant tissue are derived from a cactaceae plant, a fabaceae plant, a rutaceae plant, a podeceae plant, a malpighiaceae plant, a myristicaceae plant, or a combination thereof. In some embodiments, the plant cells or plant tissue are derived from a plant selected from Dictyoloma incanescens, Limonia acidissima, Melicope leptococca, Anadenanthera peregrina, Acacia auriculiformis, Acacia Victoriae, Desmodium gangeticum, Lespedeza bicolor, Mimosa pudica, Mucuna pruriens, Phyllodium pulchellum, Phalaris tuberosa, Banisteriopsis caapi, Diplopterys cabrerana, Echinocereus salm-dyckianus, Echinocereus triglochidiatus, Horsfieldia superba, Iryanthera macrophylla, Osteophloeum platyspermum, V. theiodora, V. calophylla, V. multinervia, V. peruviana, V. rufula, V. venosa(“disclosed exemplary plant cells and plant tissues”), or a combination thereof.

In some embodiments, the 5-MeO-DMT or salt thereof is not derived from a natural source. For example, in some embodiments, the 5-MeO-DMT or salt thereof is not extracted or derived from a plant tissue, a fungal tissue, or an animal tissue (e.g., toad venom). In embodiments, the 5-MeO-DMT or salt thereof is produced by chemical synthesis, such as, for example, multi-step chemical synthesis. In embodiments, the 5-MeO-DMT or salt thereof substantially free of any impurity selected from animal cells, animal tissue, fungal cells, fungal tissue, plant cells, and plant tissue. In some embodiments, the 5-MeO-DMT or salt thereof comprises less than, e.g., 0.01%, 0.1%, 0.2%, 0.3%, 0.4%, 0.5%, 0.6%, 07%, 0.8%, 0.8%, or 1% of an impurity selected from animal cells, animal tissue, fungal cells, fungal tissue, plant cells, and plant tissue. In embodiments, the 5-MeO-DMT or salt thereof comprises less than 1% of an impurity selected from animal cells, animal tissue, fungal cells, fungal tissue, plant cells, and plant tissue.

In some embodiments, 50% or less of the pharmaceutical composition comprises 5-MeO-DMT or salt thereof. In some embodiments, 25% or less of the pharmaceutical composition comprises 5-MeO-DMT or salt thereof. In some embodiments, 10% or less of the pharmaceutical composition comprises 5-MeO-DMT or salt thereof. In embodiments, 75% or less, 50% or less, 25% or less, 10% or less, or 5% or less of the pharmaceutical composition comprises 5-MeO-DMT or salt thereof. In embodiments, at least 80%, at least 85%, at least 90%, at least 95%, or at least 99% of the pharmaceutical composition comprises 5-MeO-DMT or salt thereof.

c. Treating Substance Use Disorder Based on 5-MeO-DMT Treatment Holiday

In some aspects the present disclosure provides methods of treating substance use disorders based on a designated 5-MeO-DMT drug holiday. In some embodiments, the drug holiday or “treatment holiday” is an interval of time that separates sessions of 5-MeO-DMT administration. In another embodiment, the method of treating an substance use disorder in a subject in need thereof comprises administering to the subject a formulation comprising between 4 mg and 40 mg of 5-MeO-DMT or a salt thereof, wherein the formulation is selected from an intramuscular formulation, an intravenous formulation, and a buccal formulation. The administration is then followed by a treatment holiday of 1 week to 4 months following said administering with the 5-MeO-DMT or salt thereof.

In some embodiments, the 5-MeO-DMT treatment holiday is from 1 week to 12 months. In some embodiments, the 5-MeO-DMT treatment holiday is from 1 week to 11 months. In some embodiments, the 5-MeO-DMT treatment holiday is from 1 week to 10 months. In some embodiments, the 5-MeO-DMT treatment holiday is from 1 week to 9 months. In some embodiments, the 5-MeO-DMT treatment holiday is from 1 week to 8 months. In some embodiments, the 5-MeO-DMT treatment holiday is from 1 week to 7 months. In some embodiments, the 5-MeO-DMT treatment holiday is from 1 week to 6 months. In some embodiments, the 5-MeO-DMT treatment holiday is from 1 week to 5 months. In some embodiments, the 5-MeO-DMT treatment holiday is from 1 week to 4 months. In some embodiments, the 5-MeO-DMT treatment holiday is from 1 week to 3 months. In some embodiments, the 5-MeO-DMT treatment holiday is from 2 weeks to 12 months. In some embodiments, the 5-MeO-DMT treatment holiday is from 2 weeks to 11 months. In some embodiments, the 5-MeO-DMT treatment holiday is from 2 weeks to 10 months. In some embodiments, the 5-MeO-DMT treatment holiday is from 2 weeks to 9 months. In some embodiments, the 5-MeO-DMT treatment holiday is from 2 weeks to 8 months. In some embodiments, the 5-MeO-DMT treatment holiday is from 2 weeks to 7 months. In some embodiments, the 5-MeO-DMT treatment holiday is from 2 weeks to 6 months. In some embodiments, the 5-MeO-DMT treatment holiday is from 2 weeks to 5 months. In some embodiments, the 5-MeO-DMT treatment holiday is from 2 weeks to 4 months. In some embodiments, the 5-MeO-DMT treatment holiday is from 2 weeks to 3 months. In some embodiments, the 5-MeO-DMT treatment holiday is from 3 weeks to 12 months. In some embodiments, the 5-MeO-DMT treatment holiday is from 4 weeks to 12 months.

In some embodiments, the 5-MeO-DMT treatment holiday is from 1 month to 12 months. In some embodiments, the 5-MeO-DMT treatment holiday is from 3 months to 12 months. In some embodiments, the 5-MeO-DMT treatment holiday is from 4 months to 12 months. In some embodiments, the 5-MeO-DMT treatment holiday is from 5 months to 12 months. In some embodiments, the 5-MeO-DMT treatment holiday is from 6 months to 12 months. In some embodiments, the 5-MeO-DMT treatment holiday is from 7 months to 12 months. In some embodiments, the 5-MeO-DMT treatment holiday is from 8 months to 12 months. In some embodiments, the 5-MeO-DMT treatment holiday is from 9 months to 12 months. In some embodiments, the 5-MeO-DMT treatment holiday is from 10 months to 12 months. In some embodiments, the 5-MeO-DMT treatment holiday is from 11 months to 12 months. In some embodiments, the 5-MeO-DMT treatment holiday is 1 week, 2 weeks, 3 weeks, 4 weeks, 1 month, 2 months, 3 months, 4 months, 5 months, 6 months, 7 months, 8 months, 9 months, 10 months, 11 months, or 12 months.

In some embodiments, the intramuscular, intravenous, sublingual, or buccal formulation comprises an acid addition salt of 5-MeO-DMT selected from the HCl addition salt and sulfuric acid addition salt. In some embodiments, the intramuscular, intravenous, sublingual, or buccal formulation comprises 5-MeO-DMT HCl.

In some embodiments, a method of treating a substance use disorder in a subject in need thereof comprises administering to the subject a pharmaceutical composition comprising 5-MeO-DMT or a salt thereof; and providing the subject with a therapy session between 1 week and 10 weeks after the administering of 5-MeO-DMT or a salt thereof. In some embodiments, the formulation administered to the subject comprises from about 4 mg to about 60 mg, about 4 mg to about 50 mg, about 4 mg to about 40 mg, about 4 mg to about 30 mg, about 4 mg to about 20 mg, or about 4 mg to about 10 mg of 5-MeO-DMT or salt thereof. In some embodiments, the pharmaceutical composition administered to the subject comprises from about 8 mg to about 60, about 8 mg to about 50 mg, about 8 mg to about 40 mg, about 8 mg to about 30 mg, about 8 mg to about 20 mg, or about 8 mg to about 16 mg of 5-MeO-DMT or salt thereof. In embodiments, the pharmaceutical composition administered to the subject comprises from about 12 mg to about 60 mg, about 12 mg to about 50 mg, or about 12 mg to about 40 mg of 5-MeO-DMT or salt thereof.

In some embodiments, the pharmaceutical composition administered to the subject comprises at least 5 mg, at least 10 mg, at least 15 mg, at least 20 mg, at least 25 mg, at least 30 mg, or at least 40 mg of 5-MeO-DMT or a salt thereof. In some embodiments, the pharmaceutical composition administered to the subject comprises at most 80 mg, at most 70 mg, at most 60 mg, at most 50 mg, at most 45 mg, at most 40 mg, or at most 35 mg of 5-MeO-DMT or a salt thereof.

In some embodiments, the total dose of 5-MeO-DMT or salt thereof administered to a subject is from about 25 mg to about 80 mg, about 25 mg to 75 mg, about 25 mg to 70 mg, about 25 mg to 65 mg, about 25 mg to 60 mg, about 25 mg to 55 mg, about 25 mg to about 50 mg, about 25 mg to about 45 mg, or about 25 mg to about 30 mg. In some embodiments, the total dose of 5-MeO-DMT or salt thereof administered to a subject is from about 35 mg to about 80 mg, about 35 mg to about 75 mg, about 35 mg to about 70 mg, about 35 mg to about 65 mg, about 35 mg to about 60 mg, or about 25 mg to about 55 mg, about 35 mg to about 50 mg, about 35 mg to about 45 mg, or about 35 mg to about 40 mg.

In some embodiments, the formulation is selected from an intramuscular formulation, an intravenous formulation, and a buccal formulation. In some embodiments, the formulation is an intramuscular formulation. In some embodiments, the formulation is an intravenous formulation. In some embodiments, the formulation is a buccal formulation.

In some embodiments, the administration of 5-MeO-DMT or salt thereof is performed by a medical professional. In some embodiments, the administration of 5-MeO-DMT or salt thereof is performed by a licensed healthcare professional. In some embodiments, the administration of 5-MeO-DMT or salt thereof is performed by a licensed medical professional.

In some embodiments, 5-MeO-DMT is administered together with psychotherapy, such as psychosocial or behavioral therapy, including any of (or adapted from any of) cognitive behavioral therapy (e.g., as described in Arch. Gen. Psychiatry 1999; 56:493-502), interpersonal therapy (e.g., as described in Psychol Addict Behav 2009; 23 (1): 168-174), contingency management based therapy (e.g., as described in Psychol Addict Behav 2009; 23 (1): 168-174; in J. Consul. Clin. Psychol. 2005; 73 (2): 354-59; or in Case Reports in Psychiatry, Vol. 2012, Article ID 731638), motivational interviewing based therapy (e.g., as described in J. Consul. Clin. Psychol. 2001; 69 (5): 858-62), or meditation based therapy, such as transcendental meditation based therapy (e.g., as described in J. Consul. Clin. Psychol. 2000; 68 (3): 515-52).

In some embodiments, the therapy session comprises psychosocial therapy. In some embodiments, the therapy session comprises a practitioner and at least one subject. In some embodiments, the therapy session is a group session with the plurality of subjects. In some embodiments, the group session comprises the practitioner and at least two subjects. In some embodiments, the therapy session occurs during the 5-MeO-DMT treatment holiday. In some embodiments, the therapy session occurs during the 5-MeO-DMT treatment holiday and comprises a practitioner and a plurality of subjects. In some embodiments, the therapy session comprises a practitioner and a plurality of subjects. In some embodiments, the practitioner is a trained medical professional. For example, the trained medical professional can include but is not limited to psychiatrists, psychologists, addiction specialists, substance abuse counselors, and therapists. In some embodiments, the trained medical profession is a psychiatrist, a psychologist, an addiction specialist, a substance abuse counselor, a therapist, or any combination thereof. In some embodiments, the therapy session comprises the practitioner and at least one subject.

In some embodiments, the therapy session occurs between 1 week and 10 weeks after the administration of the 5-MeO-DMT or salt thereof. In some embodiments, the therapy session occurs between 2 weeks and 10 weeks after the administration of the 5-MeO-DMT or salt thereof. In some embodiments, the therapy session occurs between 3 weeks and 10 weeks after the administration of the 5-MeO-DMT or salt thereof. In some embodiments, the therapy session occurs between 4 weeks and 10 weeks after the administration of the 5-MeO-DMT or salt thereof. In some embodiments, the therapy session occurs between 1 week and 8 weeks after the administration of the 5-MeO-DMT or salt thereof. In some embodiments, the therapy session occurs between 2 weeks and 8 weeks after the administration of the 5-MeO-DMT or salt thereof. In some embodiments, the therapy session occurs between 3 weeks and 8 weeks after the administration of the 5-MeO-DMT or salt thereof. In some embodiments, the therapy session occurs between 4 weeks and 8 weeks after the administration of the 5-MeO-DMT or salt thereof. In some embodiments, the therapy session occurs at least 1 day after administration of the 5-MeO-DMT or salt thereof. In some embodiments, the therapy session occurs at least, for example, 2 days, 3 days, 4 days, 5 days, 6 days, 7 days, 8 days, 9 days, or 10 days after administration of the 5-MeO-DMT or salt thereof. In some embodiments, the therapy session occurs at least, for example, 1 week, 2 weeks, 3 weeks, 4 weeks, 5 weeks, or 6 weeks after administration of the 5-MeO-DMT or salt thereof.

In some embodiments, administering said treatment attenuates substance intake by said subject by about 20% or more as compared with the amount of substance intake before said treatment. In some embodiments, the administering said treatment attenuates substance intake by said subject by about 30%, about 40%, about 50%, about 60%, about 70%, about 80%, about 90%, or about 95% or more as compared with the amount of substance intake before said treatment. In some embodiments, the administering said treatment attenuates substance intake by said subject by at least 20%, at least 30%, at least 40%, at least 50%, at least 60%, at least 70%, at least 80%, at least 90%, or at least 95% as compared with the amount of substance intake before said treatment.

In some embodiments, 80% or more of the pharmaceutical composition comprises 5-MeO-DMT or salt thereof. In some embodiments, 90% or more of the pharmaceutical composition comprises 5-MeO-DMT or salt thereof. In some embodiments, 95% or more of the pharmaceutical composition comprises 5-MeO-DMT or salt thereof. In some embodiments, 80% or more, 85% or more, 90% or more, 95% or more, or 99% or more of the pharmaceutical composition comprises 5-MeO-DMT or salt thereof. In some embodiments, at least 80%, at least 85%, at least 90%, at least 95%, or at least 99% of the pharmaceutical composition comprises 5-MeO-DMT or salt thereof.

In some embodiments, the substance use disorder is selected from substance abuse and substance dependence. In some embodiments, the substance use disorder is selected from substance abuse. In some embodiments, the substance use disorder is selected from substance dependence. In some embodiments, the substance use disorder is a substance abuse disorder as defined under DSM-5.

In some embodiments, the substance use disorder is selected from alcohol abuse disorder, cannabis use disorder, hallucinogen use disorder, inhalant use disorder, opioid use disorder, sedatives, hypnotics, or anxiolytics use disorder, stimulant use disorder, and tobacco or nicotine use disorder. In some embodiments, the substance use disorder is alcohol abuse disorder. In some embodiments, the substance use disorder is stimulant abuse disorder. In some embodiments, the substance use disorder is opioid abuse disorder. In some embodiment, the opioid abuse disorder comprises use and/or abuse of legal opioids, including, for example, legal prescription opioids. Such legal prescription opioids include, for example, hydrocodone, oxycodone, oxymorphone, morphine, codeine, hydromorphone, tapentadol, fentanyl, methadone, and the like. In some embodiments the opioid abuse disorder comprises use and/or abuse of illegal opioids, including, for example, heroin and/or fentanyl.

In some embodiments, said subject has reduced substance consumption for at least 1, 2, 3, 4, 5, 6, or 7 days. In some embodiments, said subject has reduced substance consumption for more than 1, 2, 3, 4, 5, 6, or 7 days. In some embodiments, the subject has reduced substance consumption for 1-12 days. In some embodiments, said subject has reduced substance consumption for at least 1 day. In some embodiments, said subject has reduced substance consumption for at least 2 days. In some embodiments, said subject has reduced substance consumption for at least 3 days. In some embodiments, said subject has reduced substance consumption for at least 4 days. In some embodiments, said subject has reduced substance consumption for at least 5 days. In some embodiments, said subject has reduced substance consumption for at least 6 days. In some embodiments, said subject has reduced substance consumption for at least 7 days. In some embodiments, said subject has reduced substance consumption for more than 1 day. In some embodiments, said subject has reduced substance consumption for more than 2 days. In some embodiments, said subject has reduced substance consumption for more than 3 days. In some embodiments, said subject has reduced substance consumption for more than 4 days. In some embodiments, said subject has reduced substance consumption for more than 5 days. In some embodiments, said subject has reduced substance consumption for more than 6 days. In some embodiments, said subject has reduced substance consumption for more than 7 days.

In some embodiments, the subject has reduced substance consumption for 5-12 days prior to treatment. In some embodiments, the subject has reduced substance consumption for 5-11 days prior to treatment. In some embodiments, the subject has reduced substance consumption for 5-10 days prior to treatment. In some embodiments, the subject has reduced substance consumption for 5-9 days prior to treatment. In some embodiments, the subject has reduced substance consumption for 5-8 days prior to treatment. In some embodiments, the subject has reduced substance consumption for 5-7 days prior to treatment. In some embodiments, the subject has reduced substance consumption for 5-6 days prior to treatment. In some embodiments, the subject has reduced substance consumption for 6-12 days prior to treatment. In some embodiments, the subject has reduced substance consumption for 7-12 days prior to treatment. In some embodiments, the subject has reduced substance consumption for 8-12 days prior to treatment. In some embodiments, the subject has reduced substance consumption for 9-12 days prior to treatment. In some embodiments, the subject has reduced substance consumption for 10-12 days prior to treatment. In some embodiments, the subject has reduced substance consumption for 11-12 days prior to treatment. In some embodiments, the subject has reduced substance consumption for 1-12 days prior to treatment. In some embodiments, the subject has reduced substance consumption for 1-5 days prior to treatment. In some embodiments, the subject has reduced substance consumption for 1-4 days prior to treatment. In some embodiments, the subject has reduced substance consumption for 1-3 days prior to treatment. In some embodiments, the subject has reduced substance consumption for 1-2 days prior to treatment.

In some embodiments, the pharmaceutical composition comprises less than 5% impurities. In some embodiments, the pharmaceutical composition comprises less than 5% impurities, less than 4% impurities, less than 3% impurities, less than 2% impurities, less than 1% impurities, less than 0.5% impurities, less than 0.4% impurities, less than 0.3% impurities, less than 0.2% impurities, or less than 0.1% impurities. In some embodiments, the pharmaceutical composition comprises at most 5% impurities, at most 4% impurities, at most 3% impurities, at most 2% impurities, at most 1% impurities, at most 0.5% impurities, at most 0.4% impurities, at most 0.3% impurities, at most 0.2% impurities, or at most 0.1% impurities.

In some embodiments, the impurity is selected from animal cells, animal tissue, fungal cells, fungal tissue, plant cells, and plant tissue. In some embodiments, the impurity is derived from animal cells, animal tissue, fungal cells, fungal tissue, plant cells, or plant tissue. In some embodiments, the impurity is selected from animal cells. In some embodiments, the impurity is selected from animal tissue. In some embodiments, the animal cells or animal tissue are derived from Bufo alvarius. In some embodiments, the impurity is selected from fungal cells. In some embodiments, the impurity is selected from fungal tissue. In some embodiments, the fungal cells or fungal tissue are derived from Amanita citrina, Amanita porphyria, or a combination thereof.

In some embodiments, the 5-MeO-DMT or salt thereof is not derived from a natural source. For example, in some embodiments, the 5-MeO-DMT or salt thereof is not extracted, derived, or harvested from a plant tissue, a fungal tissue, or an animal tissue (e.g., toad venom). In some embodiments, the 5-MeO-DMT or salt thereof is produced by chemical synthesis, such as, for example, multi-step chemical synthesis. In some embodiments, the 5-MeO-DMT or salt thereof substantially free of any impurity selected from animal cells, animal tissue, fungal cells, fungal tissue, plant cells, and plant tissue. In embodiments, the 5-MeO-DMT or salt thereof comprises less than, e.g., 0.01%, 0.1%, 0.2%, 0.3%, 0.4%, 0.5%, 0.6%, 07%, 0.8%, 0.8%, or 1% of an impurity selected from animal cells, animal tissue, fungal cells, fungal tissue, plant cells, and plant tissue. In embodiments, the 5-MeO-DMT or salt thereof comprises less than 1% of an impurity selected from animal cells, animal tissue, fungal cells, fungal tissue, plant cells, and plant tissue.

d. Treating Substance Use Disorder Using a 5-MeO-DMT Drug Substance

In some aspects the present disclosure provides methods of treating substance use disorders using a 5-MeO-DMT drug substance. In another embodiment, the present disclosure provides a method of treating a substance use disorder in a subject in need thereof, comprising intramuscularly, intravenously, or buccally administering a pharmaceutical composition comprising a 5-MeO-DMT drug substance and a pharmaceutically acceptable excipient to the subject wherein the pharmaceutical composition comprises less than 5% impurities. In some embodiments, the method further comprises a therapy session with the subject at least 4 hours after administration of the 5-MeO-DMT drug substance. In some embodiments, the therapy session occurs, e.g., at least 1 day, at least 1 week, or at least 1 month after administration of the 5-MeO-DMT drug substance.

In some embodiments, the pharmaceutical composition comprises less than 5% impurities. In some embodiments, the pharmaceutical composition comprises less than 5% impurities, less than 4% impurities, less than 3% impurities, less than 2% impurities, less than 1% impurities, less than 0.5% impurities, less than 0.4% impurities, less than 0.3% impurities, less than 0.2% impurities, or less than 0.1% impurities. In some embodiments, the pharmaceutical composition comprises at most 5% impurities, at most 4% impurities, at most 3% impurities, at most 2% impurities, at most 1% impurities, at most 0.5% impurities, at most 0.4% impurities, at most 0.3% impurities, at most 0.2% impurities, or at most 0.1% impurities.

In some embodiments, the impurity is selected from animal cells, animal tissue, fungal cells, fungal tissue, plant cells, and plant tissue. In some embodiments, the impurity is derived from animal cells, animal tissue, fungal cells, fungal tissue, plant cells, or plant tissue. In some embodiments, the impurity is selected from animal cells. In some embodiments, the impurity is selected from animal tissue. In some embodiments, the animal cells or animal tissue are derived from Bufo alvarius. In some embodiments, the impurity is selected from fungal cells. In some embodiments, the impurity is selected from fungal tissue. In some embodiments, the fungal cells or fungal tissue are derived from Amanita citrina, Amanita porphyria, or a combination thereof. In some embodiments, the impurity is selected from plant cells. In some embodiments, the impurity is selected from plant tissue. In some embodiments, the plant cells or plant tissue are derived from a cactaceae plant, a fabaceae plant, a rutaceae plant, a poaceae plant, a malpighiaceae plant, a myristicaceae plant, or a combination thereof. In some embodiments, the plant cells or plant tissue are derived from a plant selected from the disclosed exemplary plant cells and plant tissues, or a combination thereof.

In some embodiments, the 5-MeO-DMT or salt thereof is not derived from a natural source. For example, in some embodiments, the 5-MeO-DMT or salt thereof is not extracted or derived from a plant tissue, a fungal tissue, or an animal tissue (e.g., toad venom). In some embodiments, the 5-MeO-DMT or salt thereof is produced by chemical synthesis, such as, for example, multi-step chemical synthesis. In some embodiments, the 5-MeO-DMT or salt thereof substantially free of any impurity selected from animal cells, animal tissue, fungal cells, fungal tissue, plant cells, and plant tissue. In embodiments, the 5-MeO-DMT or salt thereof comprises less than, e.g., 0.01%, 0.1%, 0.2%, 0.3%, 0.4%, 0.5%, 0.6%, 07%, 0.8%, 0.8%, or 1% of an impurity selected from animal cells, animal tissue, fungal cells, fungal tissue, plant cells, and plant tissue. In embodiments, the 5-MeO-DMT or salt thereof comprises less than 1% of an impurity selected from animal cells, animal tissue, fungal cells, fungal tissue, plant cells, and plant tissue.

In some embodiments, the 5-MeO-DMT treatment holiday is from 1 week to 12 months. In some embodiments, the 5-MeO-DMT treatment holiday of from 1 week to 11 months. In some embodiments, the 5-MeO-DMT treatment holiday is from 1 week to 10 months. In some embodiments, the 5-MeO-DMT treatment holiday is from 1 week to 9 months. In some embodiments, the 5-MeO-DMT treatment holiday is from 1 week to 8 months. In some embodiments, the 5-MeO-DMT treatment holiday is from 1 week to 7 months. In some embodiments, the 5-MeO-DMT treatment holiday is from 1 week to 6 months. In some embodiments, the 5-MeO-DMT treatment holiday is from 1 week to 5 months. In some embodiments, the 5-MeO-DMT treatment holiday is from 1 week to 4 months. In some embodiments, the 5-MeO-DMT treatment holiday is from 1 week to 3 months. In some embodiments, the 5-MeO-DMT treatment holiday is from 2 weeks to 12 months. In some embodiments, the 5-MeO-DMT treatment holiday of from 2 weeks to 11 months. In some embodiments, the 5-MeO-DMT treatment holiday is from 2 weeks to 10 months. In some embodiments, the 5-MeO-DMT treatment holiday is from 2 weeks to 9 months. In some embodiments, the 5-MeO-DMT treatment holiday is from 2 weeks to 8 months. In some embodiments, the 5-MeO-DMT treatment holiday is from 2 weeks to 7 months. In some embodiments, the 5-MeO-DMT treatment holiday is from 2 weeks to 6 months. In some embodiments, the 5-MeO-DMT treatment holiday is from 2 weeks to 5 months. In some embodiments, the 5-MeO-DMT treatment holiday is from 2 weeks to 4 months. In some embodiments, the 5-MeO-DMT treatment holiday is from 2 weeks to 3 months. In some embodiments, the 5-MeO-DMT treatment holiday is from 3 weeks to 12 months. In some embodiments, the 5-MeO-DMT treatment holiday is from 4 weeks to 12 months.

In some embodiments, the 5-MeO-DMT treatment holiday is from 1 month to 12 months. In some embodiments, the 5-MeO-DMT treatment holiday is from 3 months to 12 months. In some embodiments, the 5-MeO-DMT treatment holiday is from 4 months to 12 months. In some embodiments, the 5-MeO-DMT treatment holiday is from 5 months to 12 months. In some embodiments, the 5-MeO-DMT treatment holiday is from 6 months to 12 months. In some embodiments, the 5-MeO-DMT treatment holiday is from 7 months to 12 months. In some embodiments, the 5-MeO-DMT treatment holiday is from 8 months to 12 months. In some embodiments, the 5-MeO-DMT treatment holiday is from 9 months to 12 months. In some embodiments, the 5-MeO-DMT treatment holiday is from 10 months to 12 months. In some embodiments, the 5-MeO-DMT treatment holiday is from 11 months to 12 months. In some embodiments, the 5-MeO-DMT treatment holiday is 1 week, 2 weeks, 3 weeks, 4 weeks, 1 month, 2 months, 3 months, 4 months, 5 months, 6 months, 7 months, 8 months, 9 months, 10 months, 11 months, or 12 months.

In some embodiments, the therapy session comprises psychosocial therapy. In some embodiments, the therapy session comprises a practitioner and at least one subject. In some embodiments, the therapy session is a group session with the plurality of subjects. In some embodiments, the group session comprises the practitioner and at least two subjects. In some embodiments, the therapy session comprises a practitioner and a plurality of subjects. In some embodiments, the practitioner is a trained medical professional. For example, the trained medical professional can include but is not limited to psychiatrists, psychologists, addiction specialists, substance abuse counselors, and therapists. In some embodiments, the trained medical profession is a psychiatrist, a psychologist, an addiction specialist, a substance abuse counselor, a therapist, or any combination thereof. In some embodiments, the therapy session comprises the practitioner and at least one subject.

e. Treating Substance Use Disorder Based on a Treatment Plan

In certain aspects the present disclosure provides methods of treating substance use disorders based on multiple treatment steps. In some embodiments, the present disclosure provides a method of treating an substance use disorder comprising administering a first treatment to the subject, wherein the first treatment comprises administering by a plurality of intramuscular injections a first combined dose of 4 mg to 40 mg, 6 mg to 40, 8 mg to 40 mg, 10 mg to 40 mg, 12 mg to 40 mg, 14 mg to 40 mg, 16 mg to 40 mg, 18 mg to 40 mg, 20 mg to 40 mg, 22 mg to 40 mg, 24 mg to 40 mg, 26 mg to 40 mg, 28 mg to 40 mg, 30 mg to 40 mg, 32 mg to 40 mg, 34 mg to 40 mg, 36 mg to 40 mg, or 38 mg to 40 mg of 5-MeO-DMT or a salt thereof; and administering a second treatment to the subject at least four weeks after the first treatment, wherein the second treatment comprises administering by a plurality of intramuscular injections a second combined dose of 5-MeO-DMT or a salt thereof, and wherein the second combined dose is equal to or greater than the first combined dose.

In some embodiments, the method further comprises a first therapy session conducted between the first treatment and the second treatment and a second therapy session conducted after the second treatment.

In some embodiments, the method further comprises administering a preparatory therapy session before said first therapy session. In some embodiments, the preparatory therapy session involves abstaining from the administration of 5-MeO-DMT or a salt thereof. In some embodiments, the preparatory therapy session comprises psychosocial therapy. In some embodiments, the preparatory therapy session, comprises a practitioner and a plurality of subjects. In some embodiments, the practitioner is a trained medical professional. In some embodiments, the preparatory session comprises a practitioner and a plurality of subjects. In some embodiments, the preparatory therapy session comprises the practitioner and at least one subject. In some embodiments, the preparatory therapy session is a group session with the plurality of subjects. In some embodiments, the preparatory group session comprises the practitioner and at least 2 subjects. In some embodiments, the preparatory group session comprises the practitioner and a subject. In some embodiments, the subject undergoes 1, 2, 3, or 4 preparatory therapy sessions. In some embodiments, during the preparatory therapy sessions, a subject may be evaluated for a reduction in substance consumption or a reduction in substance abuse as disclosed herein.

In some embodiments, the method further comprises a preparatory therapy session prior to the identifying step. In some embodiments, the method further comprises a preparatory therapy session prior to administration of 5-MeO-DMT or a salt thereof to prepare the subject for the possible psychological and physiological effects of the administering of 5-MeO-DMT. In some embodiments, the method further comprises a preparatory therapy session prior to administration of 5-MeO-DMT or a salt thereof to prepare the subject for the possible psychological effects of 5-MeO-DMT. In some embodiments, the method further comprises a preparatory therapy session prior to administration of 5-MeO-DMT or a salt thereof to prepare the subject for the possible physiological effects of the administering of 5-MeO-DMT. In some embodiments, the preparatory therapy session is provided by a licensed healthcare professional. In some embodiments, the preparatory therapy session comprises a set of instructions listing the psychological and physiological effects of the administration of 5-MeO-DMT or a salt thereof. In some embodiments, the preparatory therapy session comprises a provision of informed written consent by the subject.

In some embodiments, method further comprises one or more maintenance treatments comprising intramuscular injection of 5-MeO-DMT or a salt thereof to the subject and providing a maintenance therapy session after the treatment. In some embodiments, the method further comprises two or more maintenance treatments comprising intramuscular injection of 5-MeO-DMT or a salt thereof to the subject and providing a maintenance therapy session after the treatments.

In some embodiments, the first therapy session, the second therapy session, and the maintenance therapy session comprise practitioner and at least one subject. In some embodiments, the first therapy session, the second therapy session, and the maintenance therapy session comprise psychosocial therapy. In some embodiments, the first therapy session, the second therapy session, and the maintenance therapy session comprise a practitioner and at least one subject. In some embodiments, the first therapy session, the second therapy session, and the maintenance therapy session comprise a group session with the plurality of subjects. In some embodiments, the group session comprises the practitioner and at least two subjects. In some embodiments, the first therapy session, the second therapy session, and the maintenance therapy session comprise a practitioner and a plurality of subjects. In some embodiments, the first therapy session, the second therapy session, and the maintenance therapy session psychosocial therapy. In some embodiments, the practitioner is a trained medical professional. For example, the trained medical professional can include but is not limited to psychiatrists, psychologists, addiction specialists, substance abuse counselors, and therapists. In some embodiments, the trained medical profession is a psychiatrist, a psychologist, an addiction specialist, a substance abuse counselor, a therapist, or any combination thereof. In embodiments, the therapy session comprises the practitioner and at least one subject.

In some embodiments, the first therapy session comprises a practitioner and a plurality of subjects. In some embodiments, the practitioner is a trained medical professional. In some embodiments, the first therapy session comprises a practitioner and a plurality of subjects. In some embodiments, the first therapy session comprises the practitioner and at least one subject. In some embodiments, the first therapy session is a group session with the plurality of subjects. In some embodiments, the first group session comprises the practitioner and at least 2 subjects. In some embodiments, the first therapy session comprises psychosocial therapy.

In some embodiments, the second therapy session, comprises a practitioner and a plurality of subjects. In some embodiments, the practitioner is a trained medical professional. In some embodiments, the second therapy session comprises a practitioner and a plurality of subjects. In some embodiments, the second therapy session comprises the practitioner and at least one subject. In some embodiments, the second therapy session is a group session with the plurality of subjects. In some embodiments, the second group session comprises the practitioner and at least 2 subjects. In some embodiments, the second therapy session comprises psychosocial therapy.

In some embodiments, the maintenance therapy session comprises a practitioner and a plurality of subjects. In some embodiments, the practitioner is a trained medical professional. In some embodiments, the maintenance therapy session comprises a practitioner and a plurality of subjects. In some embodiments, the maintenance therapy session comprises the practitioner and at least one subject. In some embodiments, the maintenance therapy session is a group session with the plurality of subjects. In some embodiments, the maintenance group session comprises the practitioner and at least 2 subjects. In some embodiments, the maintenance therapy session comprises psychosocial therapy.

In some embodiments, the pharmaceutical composition comprises less than 5% impurities. In some embodiments, the pharmaceutical composition comprises less than 5% impurities, less than 4% impurities, less than 3% impurities, less than 2% impurities, less than 1% impurities, less than 0.5% impurities, less than 0.4% impurities, less than 0.3% impurities, less than 0.2% impurities, or less than 0.1% impurities. In some embodiments, the pharmaceutical composition comprises at most 5% impurities, at most 4% impurities, at most 3% impurities, at most 2% impurities, at most 1% impurities, at most 0.5% impurities, at most 0.4% impurities, at most 0.3% impurities, at most 0.2% impurities, or at most 0.1% impurities.

In some embodiments, the impurity is selected from animal cells, animal tissue, fungal cells, fungal tissue, plant cells, and plant tissue. In some embodiments, the impurity is derived from animal cells, animal tissue, fungal cells, fungal tissue, plant cells, or plant tissue. In some embodiments, the impurity is selected from animal cells. In some embodiments, the impurity is selected from animal tissue. In some embodiments, the animal cells or animal tissue are derived from Bufo alvarius. In some embodiments, the impurity is selected from fungal cells. In some embodiments, the impurity is selected from fungal tissue. In some embodiments, the fungal cells or fungal tissue are derived from Amanita citrina, Amanita porphyria, or a combination thereof. In some embodiments, the impurity is selected from plant cells. In some embodiments, the impurity is selected from plant tissue. In some embodiments, the plant cells or plant tissue are derived from a cactaceae plant, a fabaceae plant, a rutaceae plant, a poaceae plant, a malpighiaceae plant, a myristicaceae plant, or a combination thereof. In some embodiments, the plant cells or plant tissue are derived from a plant selected from the disclosed exemplary plant cells and plant tissues, or a combination thereof.

In some embodiments, the 5-MeO-DMT or salt thereof is not extracted, derived, or harvested from a natural source. For example, in some embodiments, the 5-MeO-DMT or salt thereof is not extracted, derived, or harvested from a plant tissue, a fungal tissue, or an animal tissue (e.g., toad venom). In some embodiments, the 5-MeO-DMT or salt thereof is produced by chemical synthesis, such as, for example, multi-step chemical synthesis. In some embodiments, the 5-MeO-DMT or salt thereof substantially free of any impurity selected from animal cells, animal tissue, fungal cells, fungal tissue, plant cells, and plant tissue. In some embodiments, the 5-MeO-DMT or salt thereof comprises less than, e.g., 0.01%, 0.1%, 0.2%, 0.3%, 0.4%, 0.5%, 0.6%, 07%, 0.8%, 0.8%, or 1% of an impurity selected from animal cells, animal tissue, fungal cells, fungal tissue, plant cells, and plant tissue. In some embodiments, the 5-MeO-DMT or salt thereof comprises less than 1% of an impurity selected from animal cells, animal tissue, fungal cells, fungal tissue, plant cells, and plant tissue.

In some embodiments, administering said treatment attenuates substance intake by said subject by about 20% or more as compared with the amount of substance intake before said treatment. In some embodiments, the administering said treatment attenuates substance intake by said subject by about 30%, about 40%, about 50%, about 60%, about 70%, about 80%, about 90%, or about 95% or more as compared with the amount of substance intake before said treatment. In some embodiments, the administering said treatment attenuates substance intake by said subject by at least 20%, at least 30%, at least 40%, at least 50%, at least 60%, at least 70%, at least 80%, at least 90%, or at least 95% as compared with the amount of substance intake before said treatment.

In some embodiments, 80% or more of the pharmaceutical composition comprises 5-MeO-DMT or salt thereof. In some embodiments, 90% or more of the pharmaceutical composition comprises 5-MeO-DMT or salt thereof. In some embodiments, 95% or more of the pharmaceutical composition comprises 5-MeO-DMT or salt thereof. In some embodiments, 80% or more, 85% or more, 90% or more, 95% or more, or 99% or more of the pharmaceutical composition comprises 5-MeO-DMT or salt thereof. In some embodiments, at least 80%, at least 85%, at least 90%, at least 95%, or at least 99% of the pharmaceutical composition comprises 5-MeO-DMT or salt thereof.

In some embodiments, the subject is instructed to refrain from eating food for 1 to 24 hours prior to administration of 5-MeO-DMT or a salt thereof. In some embodiments, the subject is instructed to refrain from eating food for 2 to 24 hours prior to administration of 5-MeO-DMT or a salt thereof. In some embodiments, the subject is instructed to refrain from eating food for 3 to 24 hours prior to the administration of 5-MeO-DMT or a salt thereof. In some embodiments, the subject is instructed to refrain from eating food for 4 to 24 hours prior to the administration of 5-MeO-DMT or a salt thereof. In some embodiments, the subject is instructed to refrain from eating food for 5 to 24 hours prior to the administration of 5-MeO-DMT or a salt thereof. In some embodiments, the subject is instructed to refrain from eating food for 6 to 24 hours prior to the administration of 5-MeO-DMT or a salt thereof. In some embodiments, the subject is instructed to refrain from eating food for 7 to 24 hours prior to the administration of 5-MeO-DMT or a salt thereof. In some embodiments, the subject is instructed to refrain from eating food for 8 to 24 hours prior to the administration of 5-MeO-DMT or a salt thereof. In embodiments, the subject is instructed to refrain from eating food for 9 to 24 hours prior to the administration of 5-MeO-DMT or a salt thereof. In some embodiments, the subject is instructed to refrain from eating food for 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours prior to the administration of 5-MeO-DMT or a salt thereof.

In some embodiments, the subject is instructed to refrain from eating food for at least 1 to 10 hours prior to the administration of 5-MeO-DMT or a salt thereof. In some embodiments, the subject is instructed to refrain from eating food for at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours prior to the administration of 5-MeO-DMT or a salt thereof. In some embodiments, the subject is instructed to refrain from eating food for at least 10 hours prior to the administration of 5-MeO-DMT or a salt thereof. In some embodiments, the subject is instructed to refrain from eating food for at least 12 hours prior to the administration of 5-MeO-DMT or a salt thereof. In some embodiments, the subject is instructed to refrain from eating food for at least 24 hours prior to the administration of 5-MeO-DMT or a salt thereof.

In some embodiments, physiological monitoring of the subject accompanies administration of 5-MeO-DMT or a salt thereof. In some embodiments, the physiological monitoring of the subject comprises monitoring of a physiological parameter selected from supine blood pressure, pulse rate, respiratory rate, and body temperature. In some embodiments, the physiological parameter is supine blood pressure. In some embodiments, the physiological parameter is the pulse rate. In some embodiments, the physiological parameter is the respiratory rate. In some embodiments, the physiological parameter is the body temperature.

In some embodiments, the physiological monitoring of the subject comprises monitoring of a plasma biomarker selected from brain-derived neurotrophic factor (BDNF), hypothalamic-pituitary-adrenal (HPA)-axis endocrine measures, and neuroendocrine measures. In some embodiments, the plasma biomarker is a brain-derived neurotrophic factor (BDNF). In some embodiments, the plasma biomarker is a hypothalamic-pituitary-adrenal (HPA)-axis endocrine measure. In some embodiments, the plasma biomarker is a neuroendocrine measure. In some embodiments, a plasma biomarker analysis is conducted before the dosing and after the dosing. In some embodiments, a plasma biomarker analysis is conducted before the dosing or after the dosing. In some embodiments, a plasma biomarker analysis is conducted before the dosing. In some embodiments, a plasma biomarker analysis is conducted after the dosing. In some embodiments, the plasma biomarker is drawn at 0.5 hour, 1 hour, 1.5 hours, 2 hours, 2.5 hours, 3 hours, 4 hours, 6 hours, 8 hours, 12 hours, 24 hours, or 48 hours before the dosing. In some embodiments, the plasma biomarker is drawn at 0.5 hour, 1 hour, 1.5 hours, 2 hours, 2.5 hours, 3 hours, 4 hours, 6 hours, 8 hours, 12 hours, 24 hours, or 48 hours after the dosing. In some embodiments, additional plasma biomarker analysis may be conducted if the subject has an adverse effect.

In some embodiments, administration of 5-MeO-DMT or a salt thereof further comprises psychological monitoring of the subject. In some embodiments, the psychological monitoring of the subject comprises monitoring for negative psychological reactions to treatment. In some embodiments, the psychological monitoring of the subject comprises monitoring changes in mood in the subject. In some embodiments, the changes in mood are evaluated using questionnaires. In some embodiments, the changes in mood are evaluated using questionnaires administered by the subject. In some embodiments, the changes in mood evaluated using questionnaire selected from a Psychological Insight Questionnaire (PIQ), Psychological Insight Scale (PIS), Challenging Experience Questionnaire (CEQ), Emotional Breakthrough Inventory (EBI), and Persisting Effects Questionnaire (PEQ).

In some embodiments, administration of 5-MeO-DMT or a salt thereof is monitored by a licensed healthcare professional. In some embodiments, the licensed healthcare professional is selected from a medical doctor, a psychiatrist, a clinical psychologist, a counseling psychologist, a nurse practitioner, a physician assistant, a registered nurse, a clinical scientist, an addiction specialist, a substance abuse counselor, and a therapist. In some embodiments, administration of 5-MeO-DMT or a salt thereof is monitored by a medical doctor. In some embodiments, administration of 5-MeO-DMT or a salt thereof is monitored by a psychiatrist. In some embodiments, administration of 5-MeO-DMT or a salt thereof is monitored by a clinical psychologist. In some embodiments, administration of 5-MeO-DMT or a salt thereof is monitored by a counseling psychologist. In some embodiments, administration of 5-MeO-DMT is monitored by a nurse practitioner. In some embodiments, administration of 5-MeO-DMT or a salt thereof is monitored by a physician assistant. In some embodiments, administration of 5-MeO-DMT or a salt thereof is monitored by a registered nurse. In some embodiments, administration of 5-MeO-DMT or a salt thereof is monitored by a clinical scientist. In some embodiments, administration of 5-MeO-DMT or a salt thereof is monitored by an addiction specialist. In some embodiments, administration of 5-MeO-DMT or a salt thereof is monitored by a substance abuse counselor. In some embodiments, administration of 5-MeO-DMT or a salt thereof is monitored by a therapist.

In some embodiments, the method further comprises a follow-up consultation after administration of 5-MeO-DMT. In some embodiments, the follow-up consultation may be conducted by telephone or as a video call visit or as in-clinic visit. In some embodiments, the follow-up visit is conducted by a licensed healthcare professional. In some embodiments, the follow-up consultation comprises psychosocial care or an evaluation. In some embodiments, the follow-up consultation comprises psychosocial care. In some embodiments, the psychosocial care is conducted by a licensed healthcare professional. In some embodiments, the follow-up consultation comprises an evaluation. In some embodiments, the evaluation is conducted by a licensed healthcare professional. In some embodiments, the evaluation comprises a questionnaire, a post experience evaluation, a breakthrough evaluation, or an additional therapy evaluation. In some embodiments, the evaluation is self-administered by the subject. In some embodiments, the evaluation comprises a questionnaire. In some embodiments, the questionnaire is self-administered by the subject. In some embodiments, the evaluation is selected from a Psychological Insight Questionnaire (PIQ), Psychological Insight Scale (PIS), Challenging Experience Questionnaire (CEQ), Emotional Breakthrough Inventory (EBI), Persisting Effects Questionnaire (PEQ), hallucinogenic Rating Scale (HRS), Mystical Experience Questionnaire 30-item (MEQ-30), 5-Dimensional Altered States of Consciousness Rating Scale (5D-ASC), and Warwick-Edinburgh Mental Well-Being Scale (WEMWBS).

In some embodiments, the follow-up consultation is up to 1 day, 2 days, 3 days, 4 days, 5 days, 6 days, 7 days, 14 days, 21 days, 28 days or 30 days after administration of 5-MeO-DMT. In some embodiments, the follow-up consultation is between 1 to 7 days after administration of 5-MeO-DMT. In some embodiments, the follow-up consultation is between 1 to 6 days after administration of 5-MeO-DMT. In some embodiments, the follow up consultation is between 1 to 5 days after administration of 5-MeO-DMT. In some embodiments, the follow-up consultation is between 1 to 4 days after administration of 5-MeO-DMT. In some embodiments, the follow-up consultation is between 1 to 3 days after administration of 5-MeO-DMT. In some embodiments, the follow-up consultation is 1 day after administration of 5-MeO-DMT. In some embodiments, the follow-up consultation is 2 days after administration of 5-MeO-DMT. In some embodiments, the follow-up consultation is 3 days after administration of 5-MeO-DMT. In some embodiments, the follow-up consultation is 4 days after administration of 5-MeO-DMT. In some embodiments, the follow-up consultation is 5 days after administration of 5-MeO-DMT. In some embodiments, the follow-up consultation is 6 days after administration of 5-MeO-DMT. In some embodiments, the follow-up consultation is 7 days after administration of 5-MeO-DMT. In some embodiments, the follow-up consultation is conducted by a licensed healthcare professional.

In some embodiments, the subject has refrained from using a psychedelic drug for 6 months, 5 months, 4 months, 3 months, 2 months, or 1 month before administration of 5-MeO-DMT. In some embodiments, the subject has refrained from using a psychedelic drug for 6 months before administration of 5-MeO-DMT. In some embodiments, the subject has refrained from using a psychedelic drug for 5 months before administration of 5-MeO-DMT. In some embodiments, the subject has refrained from using a psychedelic drug for 4 months before administration of 5-MeO-DMT. In some embodiments, the subject has refrained from using a psychedelic drug for 3 months before administration of 5-MeO-DMT. In some embodiments, the subject has refrained from using a psychedelic drug for 2 months before administration of 5-MeO-DMT. In some embodiments, the subject has refrained from using a psychedelic drug for 1 month before administration of 5-MeO-DMT. In some embodiments, the psychedelic drug is selected from mescaline, LSD, psilocybin, and DMT. In some embodiments, the psychedelic drug is mescaline. In some embodiments, the psychedelic drug is LSD. In some embodiments, the psychedelic drug is psilocybin. In some embodiments, the psychedelic drug is DMT.

In some embodiments, the subject is domiciled in a center during administration of 5-MeO-DMT. In some embodiments, the center is a clinic. In some embodiments, the center is an addiction treatment center. In some embodiments, the subject is domiciled at the center for at least 30 days before administration of 5-MeO-DMT. In some embodiments, the subject is domiciled at the center for at least 1 day, at least 2 days, at least 3 days, at least 4 days, at least 5 days, at least 6 days, at least 7 days, at least 14 days, at least 21 days, at least 28 days, or at least 30 days prior to during administration of 5-MeO-DMT. In some embodiments, the subject is domiciled at the center for 30 days before during administration of 5-MeO-DMT. In some embodiments, the subject is domiciled at the center for 1 day, 2 days, 3 days, 4 days, 5 days, 6 days, 7 days, 14 days, 21 days, 28 days, or 30 days prior to administration of 5-MeO-DMT. In some embodiments, the subject is domiciled for at least 30 days after administration of 5-MeO-DMT. In some embodiments, the subject is domiciled at the center for at least 1 day, at least 2 days, at least 3 days, at least 4 days, at least 5 days, at least 6 days, at least 7 days, at least 14 days, at least 21 days, at least 28 days, or at least 30 days after administration of 5-MeO-DMT. In some embodiments, the subject is domiciled at the center for 30 days before administration of 5-MeO-DMT. In some embodiments, the subject is domiciled at the center for 1 day, 2 days, 3 days, 4 days, 5 days, 6 days, 7 days, 14 days, 21 days, 28 days, or 30 days after administration of 5-MeO-DMT. In some embodiments, the subject is located outside of a center for administration of 5-MeO-DMT.

### C. Pharmaceutical Compositions and Formulations

In certain aspects, the present disclosure provides a pharmaceutical composition comprising at least one pharmaceutically acceptable excipient and 5-MeO-DMT or salt thereof. “Pharmaceutical compositions” are compositions that include the disclosed compound(s) together in an amount (for example, in a unit dosage form) with a pharmaceutically acceptable carrier, diluent, or excipient. Some embodiments will not have a single carrier, diluent, or excipient alone, but will include multiple carriers, diluents, and/or excipients. Compositions can be prepared by standard pharmaceutical formulation techniques such as disclosed in, e.g., Remington: The Science & Practice of Pharmacy (2020) 23th ed., Academic Press., Cambridge, Mass.; The Merck Index (1996) 12th ed., Merck Pub. Group, Whitehouse, N.J.; Pharm. Principles of Solid Dosage Forms (1993), Technomic Pub. Co., Inc., Lancaster, Pa.; and Ansel & Stoklosa, Pharm. Calculations (2001) 11th ed., Lippincott Williams & Wilkins, Baltimore, Md.; & Poznansky et al. Drug Delivery Systems (1980), R.L. Juliano, ed., Oxford, N.Y., pp. 253-315).

“Pharmaceutically acceptable” used in connection with an excipient, carrier, diluent, or other ingredient means the ingredient is generally safe and, within the scope of sound medical judgment, suitable for use in contact with cells of humans and animals without undue toxicity, irritation, allergic response, or complication, commensurate with a reasonable risk/benefit ratio.

In some embodiments, the pharmaceutical composition is formulated into a pharmaceutical formulation. Pharmaceutical formulations may be provided in any suitable form, which may depend on the route of administration. In some embodiments, the pharmaceutical composition disclosed herein can be formulated in dosage form for administration to a subject.

In some embodiments, the pharmaceutical composition is formulated for sublingual, intravenous, intraarterial, aerosol, parenteral, buccal, topical, transdermal, rectal, intramuscular, subcutaneous, intraosseous, intranasal, intrapulmonary, transmucosal, inhalation, oral, and/or intraperitoneal administration.

In some embodiments parenteral administration characterized by injection, either subcutaneously, intramuscularly, or intravenously is also contemplated herein. In some embodiments, the pharmaceutical composition is formulated for intramuscular injection. In some embodiments, the pharmaceutical composition is formulated for intravenous injection. Injectables can be prepared in conventional forms, either as liquid solutions or suspensions, solid forms suitable for solution or suspension in liquid prior to injection, or as emulsions. In some embodiments, the pharmaceutical composition disclosed herein can be formulated for buccal or sublingual administration.

In some embodiments, the 5-MeO-DMT is administered as a pharmaceutically acceptable salt, as expressed by the phrase “5-MeO-DMT or a salt thereof” as expressed in various embodiments herein. The term “salt” or “pharmaceutically acceptable salt” refers to a salt prepared from pharmaceutically acceptable non-toxic acids or bases, and which may be synthesized by conventional chemical methods. Generally, such salts are prepared by reacting the free acid or base forms of these agents with a stoichiometric amount of the appropriate base or acid in water or in an organic solvent, or in a mixture of the two; generally, nonaqueous media (e.g., ether, ethyl acetate, ethanol, isopropanol, or acetonitrile) are preferred. For therapeutic use, salts of 5-MeO-DMT are those wherein the counter-ion is pharmaceutically acceptable.

Exemplary salts include 2-hydroxyethanesulfonate, 2-naphthalenesulfonate, 2-napsylate, 3-hydroxy-2-naphthoate, 3-phenylpropionate, 4-acetamidobenzoate, acefyllinate, acetate, aceturate, adipate, alginate, aminosalicylate, ammonium, amsonate, ascorbate, aspartate, benzenesulfonate, benzoate, besylate, bicarbonate, bisulfate, bitartrate, borate, butyrate, calcium edetate, calcium, camphocarbonate, camphorate, camphorsulfonate, camsylate, carbonate, cholate, citrate, clavulariate, cyclopentanepropionate, cypionate, d-aspartate, d-camsylate, d-lactate, decanoate, dichloroacetate, digluconate, dodecylsulfate, edentate, edetate, edisylate, estolate, esylate, ethanesulfonate, ethyl sulfate, fumarate, furate, fusidate, galactarate (mucate), galacturonate, gallate, gentisate, gluceptate, glucoheptanoate, gluconate, glucuronate, glutamate, glutarate, glycerophosphate, glycolate, glycollylarsanilate, hemisulfate, heptanoate (enanthate), heptanoate, hexafluorophosphate, hexanoate, hexylresorcinate, hippurate, hybenzate, hydrabamine, hydrobromide, hydrobromide/bromide, hydrochloride, hydroiodide, hydroxide, hydroxybenzoate, hydroxynaphthoate, iodide, isethionate, isothionate, 1-aspartate, 1-camsylate, 1-lactate, lactate, lactobionate, laurate, laurylsulphonate, lithium, magnesium, malate, maleate, malonate, mandelate, meso-tartrate, mesylate, methanesulfonate, methylbromide, methylnitrate, methylsulfate, mucate, myristate, N-methylglucamine ammonium salt, napadisilate, naphthylate, napsylate, nicotinate, nitrate, octanoate, oleate, orotate, oxalate, p-toluenesulfonate, palmitate, pamoate, pantothenate, pectinate, persulfate, phenylpropionate, phosphate, phosphateldiphosphate, picrate, pivalate, polygalacturonate, potassium, propionate, pyrophosphate, saccharate, salicylate, salicylsulfate, sodium, stearate, subacetate, succinate, sulfate, sulfosaliculate, sulfosalicylate, suramate, tannate, tartrate, teoclate, terephthalate, thiocyanate, thiosalicylate, tosylate, tribrophenate, triethiodide, undecanoate, undecylenate, valerate, valproate, xinafoate, zinc and the like. (See Berge et al. (1977) “Pharmaceutical Salts,” J. Pharm. Sci. 66:1-19.)

In some embodiments, the dosage form is formulated for oral administration, e.g., oral dosage form. For example, the pharmaceutical composition can be formulated in the form of a pill, a tablet, a capsule, an inhaler, a liquid suspension, a liquid emulsion, a gel, or a powder. In some embodiments, the pharmaceutical composition can be formulated as a unit dosage in liquid, gel, semi-liquid, semi-solid, or solid form. Oral dosage forms are either solid, gel or liquid. The solid dosage forms are tablets, capsules, granules, and bulk powders. Types of oral tablets include compressed, chewable lozenges and tablets which may be enteric coated, sugar coated, or film coated. Capsules may be hard or soft gelatin capsules, while granules and powders may be provided in non-effervescent or effervescent form with the combination of other ingredients known to those skilled in the art.

In some embodiments, the disclosure provides a pharmaceutical composition for buccal or sublingual administration comprising 5-MeO-DMT or a salt thereof and a pharmaceutical excipient suitable for buccal or sublingual administration. The composition may be in the form of a solid, liquid, gel, semi-liquid, or semi-solid. Pharmaceutical compositions of the disclosure suitable for buccal or sublingual administration can be presented as discrete dosage forms, such as hard or soft capsules, cachets, troches, lozenges, or tablets, or liquids or aerosol sprays. The compounds are, in some embodiments, formulated into suitable preparations such as solutions, suspensions, tablets, dispersible tablets, pills, capsules, powders, sustained release formulations or elixirs, for buccal or sublingual administration or in sterile solutions or suspensions for parenteral administration, transdermal administration and oral inhalation via nebulizers, pressurized metered dose inhalers and dry powder inhalers. In some embodiments, 5-MeO-DMT or a salt thereof may be formulated into compositions using techniques and procedures well known in the art (see, e.g., Ansel, Introduction to Pharmaceutical Dosage Forms, Seventh Edition (1999)).

In making the compositions employed in the invention the 5-MeO-DMT is usually mixed with an excipient, diluted by an excipient, or enclosed within such a carrier which can be in the form of a capsule, sachet, paper or other container. When the excipient serves as a diluent, it can be a solid, semi-solid, or liquid material, which acts as a vehicle, carrier, or medium for the active ingredient. Thus, the compositions can be in the form of tablets (including orally disintegrating, swallowable, sublingual, buccal, and chewable tablets), pills, powders, lozenges, troches, oral films, thin strips, sachets, cachets, elixirs, suspensions, emulsions, microemulsions, liposomal dispersions, aqueous and non-aqueous solutions, slurries, syrups, aerosols (as a solid or in a liquid medium), ointments containing for example up to 10% by weight of the active compound, soft and hard gelatin capsules, suppositories, topical preparations, transdermal patches, sterile injectable solutions, and sterile packaged powders. Compositions may be formulated as immediate release, controlled release, sustained (extended) release or modified release formulations. In embodiments, the composition is prepared as a dry powder for inhalation or a liquid preparation for vaporization and inhalation, and is administered, e.g., using an electronic cigarette or other vaping device, a nebulizer, a pressurized metered dose inhaler (pMDI), a dry powder inhaler (DPI), or the like.

In preparing a formulation, it may be necessary to mill a disclosed compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g., about 40 mesh.

Examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. Formulations can additionally include: lubricating agents such as talc, magnesium stearate, and mineral oil; wetting agents; emulsifying and suspending agents; preserving agents such as methyl- and propylhydroxybenzoates; sweetening agents; and flavoring agents. The disclosed compositions can be formulated so as to provide quick, sustained or delayed release of the active ingredient after administration to the patient by employing procedures known in the art.

In embodiments, the disclosed compositions are formulated in a pharmaceutically acceptable oral dosage form. Oral dosage forms include liquid dosage forms (such as tinctures, drops, emulsions, syrups, elixirs, suspensions, and solutions, and the like) and solid dosage forms.

The disclosed pharmaceutical compositions also may be prepared as formulations suitable for intramuscular, subcutaneous, intraperitoneal, or intravenous injection, comprising physiologically acceptable sterile aqueous or non-aqueous solutions, dispersions, suspensions or emulsions, liposomes, and sterile powders for reconstitution into sterile injectable solutions or dispersions. An exemplary injectable formulation may be prepared using, in one embodiment, 125 mg of 5-MeO-DMT for every 5 mL of suitable aqueous or non-aqueous carrier, diluent, solvent, or vehicle, wherein the 5-MeO-DMT is dissolved therein, optionally together with one or more preservatives, additives, or additional active agents. Examples of suitable aqueous and non-aqueous carriers, diluents, solvents, or vehicles include water, ethanol, polyols, suitable mixtures thereof, vegetable oils, and injectable organic esters such as ethyl oleate. Additionally, the disclosed compositions can be dissolved at concentrations of >1 mg/ml using water-soluble beta cyclodextrins (e.g., beta-sulfobutyl-cyclodextrin and 2-hydroxypropyl-betacyclodextrin. Proper fluidity can be maintained, for example, by the use of a coating such as a lecithin, by the maintenance of the required particle size in the case of dispersions, and by the use of surfactants.

Formulations suitable for subcutaneous injection also may contain additives such as preserving, wetting, emulsifying, and dispensing agents. Prevention of the growth of microorganisms can be ensured by various antibacterial and antifungal agents, such as parabens, benzoic acid, benzyl alcohol, chlorobutanol, phenol, and sorbic acid. Isotonic agents, such as sugars and sodium chloride may be used. Prolonged drug absorption of an injectable form can be brought about by use of agents delaying absorption, e.g., aluminum monostearate or gelatin.

Disclosed compositions also may be prepared as suspension formulations designed for extended-release via subcutaneous or intramuscular injection. Such formulations avoid first-pass metabolism, and lower dosages of the active agents will be necessary to maintain equivalent plasma levels when compared to oral formulations. In such formulations, the mean particle size of the active agents and the range of total particle sizes can be used to control the release of those agents by controlling the rate of dissolution in fat or muscle. The compositions also may be prepared for microinjection or injection cannula.

### D. Dosing

In some embodiments, where a pharmaceutical composition includes 5-MeO-DMT or a salt thereof, it may be present in an amount so that a single dose is, or that a single dose administered or able to be administered is (whether or not such dose is present in a unit dosage form), e.g., 25 mg or less (including a dose of 10 mg or less, 5 mg or less, 1 mg or less, and 0.5 mg or less), at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 105 mg, at least 110 mg, at least 115 mg, at least 120 mg, at least 125 mg, at least 130 mg, at least 135 mg, at least 140 mg, at least 145 mg, at least 150 mg, at least 155 mg, at least 160 mg, at least 165 mg, at least 170 mg, at least 175 mg, at least 180 mg, at least 185 mg, at least 190 mg, at least 195 mg, at least 200 mg, at least 225 mg, or at least 250 mg, as well as amounts within these ranges.

In some embodiments, the pharmaceutical composition administered to the subject comprises at least 5 mg, at least 10 mg, at least 15 mg, at least 20 mg, at least 25 mg, at least 30 mg, or at least 40 mg of 5-MeO-DMT or a salt thereof. In some embodiments, the pharmaceutical composition administered to the subject comprises at most 80 mg, at most 70 mg, at most 60 mg, at most 50 mg, at most 45 mg, at most 40 mg, or at most 35 mg of 5-MeO-DMT or a salt thereof. In some embodiments, the total dose of 5-MeO-DMT or a salt thereof administered to a subject is at least 5 mg, at least 10 mg, at least 15 mg, at least 20 mg, at least 25 mg, at least 30 mg, or at least 40 mg of 5-MeO-DMT or a salt thereof. In some embodiments, the total dose of 5-MeO-DMT or a salt thereof administered to a subject is at most 80 mg, at most 70 mg, at most 60 mg, at most 50 mg, at most 45 mg, at most 40 mg, or at most 35 mg of 5-MeO-DMT or a salt thereof. In some embodiments, the total dose of 5-MeO-DMT or a salt thereof administered to a subject is 6 mg to about 80 mg, 6 mg to 70 mg, 6 mg to 60 mg, 6 mg to 50 mg, 6 mg to 45 mg, 6 mg to 40 mg, or 6 mg to 35 mg, wherein each range is inclusive. In some embodiments, the total dose of 5-MeO-DMT or a salt thereof administered to a subject is 10 mg to about 80 mg, 20 mg to 80 mg, 25 mg to 80 mg, 40 mg to 50 mg, 50 mg to 80 mg, or 60 mg to 80 mg, wherein each range is inclusive. In some embodiments, the total dose of 5-MeO-DMT or a salt thereof administered to a subject is 10 mg to about 60 mg, 20 mg to 60 mg, 25 mg to 60 mg, 40 mg to 60 mg, or 50 mg to 60 mg, wherein each range is inclusive. In some embodiments, the total dose of 5-MeO-DMT or a salt thereof administered to a subject is 10 mg to about 50 mg, 20 mg to 50 mg, 25 mg to 50 mg, or 40 mg to 50 mg, wherein each range is inclusive.

In some embodiments, the dose of 5-MeO-DMT, or a plurality of doses, is administered to the subject at an interval of 1 to 10 hours, 1 to 9 hours, 1 to 8 hours, 1 to 7 hours, 1 to 6 hours, 1 to 5 hours, 1 to 4 hours, 1 to 3 hours, or 1 to 2 hours. In some embodiments, the administering is performed within a period of, for example, 2 to 10 hours, 2 to 9 hours, 2 to 8 hours, 2 to 7 hours, 2 to 6 hours, 2 to 5 hours, 2 to 4 hours, or 2 to 3 hours. In some embodiments, each of the plurality of doses is spaced apart by between 30 minutes and 2 hours. In some embodiments, each of the plurality of doses is spaced apart by between 30 minutes and 1.5 hours. In some embodiments, each of the plurality of doses is spaced apart by between 30 minutes and 1 hour. In some embodiments, each of the plurality of injection is spaced apart by between about 30 minutes, about 1 hour, about 1.5 hours, or about 2 hours.

It will be appreciated that the disclosed compositions are not limited to combinations of a single compound, or (when formulated as a pharmaceutical composition) limited to a single carrier, diluent, and/or excipient alone, but may also include combinations of multiple compounds (including additional active compounds), and/or multiple carriers, diluents, and excipients. Pharmaceutical compositions of this invention thus may comprise 5-MeO-DMT or a salt thereof together with one or more other active agents (or their derivatives and analogs) in combination, together with one or more pharmaceutically-acceptable carriers, diluents, and/or excipients, and additionally with one or more other active compounds.

In some embodiments, a formulation of the invention will be prepared so as to increase an existing therapeutic effect, provide an additional therapeutic effect, increase a desired property such as stability or shelf-life, decrease an unwanted effect or property, alter a property in a desirable way (such as pharmacokinetics or pharmacodynamics), modulate a desired system or pathway (e.g., a neurotransmitter system), or provide synergistic effects.

“Therapeutic effects” that may be increased or added in embodiments of the invention include, but are not limited to, antioxidant, anti-inflammatory, analgesic, antineuropathic, antinociceptive, antimigraine, anxiolytic, antidepressant, antipsychotic, anti-PTSD, dissociative, immunostimulant, anti-cancer, antiemetic, orexigenic, antiulcer, antihistamine, antihypertensive, anticonvulsant, antiepileptic, bronchodilator, neuroprotective, nootropic, entactogenic, empathogenic, entheogenic, psychedelic, sedative, and stimulant effects.

“Synergistic effects” should be understood to include increases in potency, bioactivity, bioaccessibility, bioavailability, or therapeutic effect, that are greater than the additive contributions of the components acting alone. Numerous methods known to those of skill in the art exist to determine whether there is synergy as to a particular effect, i.e., whether, when two or more components are mixed together, the effect is greater than the sum of the effects of the individual components when applied alone, thereby producing “1+1>2.” Suitable methods include the isobologram analysis (or contour method) (Huang, Front Pharmacol., 2019; 10:1222) and the equation of Loewe additivity (Loewe & Muischnek, 1926, Arch. Exp. Pathol Pharmacol. 114:313-326). A synergistic effect also may be calculated using methods such as the Sigmoid-Emax equation (Holford & Scheiner, 1981, Clin. Pharmacokinet. 6:429-453) and the median-effect equation (Chou & Talalay, 1984, Adv. Enzyme Regul. 22:27-55). The corresponding graphs associated with the equations referred to above are the concentration-effect curve and combination index curve, respectively. Each equation referred to above may be applied to experimental data to generate a corresponding graph to aid in assessing the effects of the drug combination.

The goal of increasing an existing therapeutic effect, providing an additional therapeutic effect, increasing a desired property such as stability or shelf-life, decreasing an unwanted effect or property, altering a property in a desirable way (such as pharmacokinetics or pharmacodynamics), modulating a desired system or pathway (e.g, a neurotransmitter system), or otherwise inducing synergy, in some embodiments is achieved by the inclusion of an additional active compound.

Such additional active compounds may be selected from the group including amino acids, antioxidants, anti-inflammatory agents, analgesics, antineuropathic and antinociceptive agents, antimigraine agents, anxiolytics, antidepressants, antipsychotics, anti-PTSD agents, cannabinoids, dissociatives, immunostimulants, anti-cancer agents, antiemetics, orexigenics, antiulcer agents, antihistamines, antihypertensives, anticonvulsants, antiepileptics, bronchodilators, neuroprotectants, nootropics, entactogens, empathogens, entheogens, psychedelics, monoamine oxidase inhibitors, tryptamines, terpenes, phenethylamines, sedatives, stimulants, serotonergic agents, and vitamins. These ingredients may be in ion, freebase, or salt form, and may be isomers, prodrugs, derivatives (preferably physiologically functional derivatives), or analogs.

In some instances, certain personalized approaches may be utilized, based on individual characteristics, including drug metabolism or individual genetic variation. The term “genetic variation” refers to a change in a gene sequence relative to a reference sequence (e.g., a commonly-found and/or wild-type sequence). Genetic variation may be recombination events or mutations such as substitution/deletion/insertion events like point and splice site mutations.

In one embodiment, the genetic variation is a genetic variation in one or more cytochrome P450 (CYP or CYP450) enzymes that affects drug metabolism, including metabolism of a disclosed composition, and including CYP1A2, CYP2C9, CYP2D6, CYP2C19, CYP3A4 and CYP3A5. Other examples of CYP enzymes include CYP1A1, CYP1B1, CYP2A6, CYP2A13, CYP2B6, CYP2C8, CYP2C9, CYP2C18, CYP2E1, CYP2G1, CYP2J2, CYP2R1, CYP2S1, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, and CYP51.

In some embodiments, a disclosed composition is taken together with a compound that is metabolized by the same CYP enzyme(s) as the disclosed composition, so as to permit a lower dose to be taken, increase the effective bioavailability of one or both, or otherwise affect drug metabolism or pharmacokinetics. In some embodiments, the dose of a disclosed composition is adjusted when administered to a subject known to be a “poor metabolizer” of 5-MeO-DMT. In embodiments, a genetic variation is an exclusion criteria for a disclosed method.

In one embodiment, the genetic variation is a genetic variation in metabotropic glutamate receptor type 5 (mGluR5), which has been implicated in mood and anxiety symptoms in humans. In another embodiment, the genetic variation is one or more single nucleotide polymorphisms (SNPs) in the FKBP5 gene that are associated with elevated levels of FKBP51 protein relative to persons lacking such SNPs. The FKBP5 gene has been implicated in responses to stress and trauma, and such SNPs are correlated with susceptibility to certain depression, PTSD, and anxiety disorders. In one embodiment, the genetic variation is a genetic variation such as a SNP in a membrane transporter, such as SERT, DAT, NET, or VMAT. In one embodiment, the mammal being treated has altered epigenetic regulation of a gene the expression of which is associated with a mental health condition or susceptibility to a mental health treatment, such as the SIGMAR1 gene for the non-opioid sigma-1 receptor.

### E. Examples

The following examples are included to further describe some embodiments of the present disclosure and should not be used to limit the scope of the disclosure.

### Example 1: Synthesis of 5-MeO-DMT

5-MeO-DMT may be synthesized using any procedure known in the art, including, for example, procedures as described in, for example, A. Sherwood, et al., Synthesis and Characterization of 5-MeO-DMT Succinate for Clinical Use, ACS Omega, 5:32067-32075 (2020); G. Bertaccini, et al., Synthesis and pharmacological activity of some 5-methoxyindole derivatives occurring in nature, Edizione Scientifica 22:229-44 (1967); or F. Benington et al., Synthesis of O- and N-methylated derivatives of 5-hydroxytryptamine, J. Am. Chem. Soc. 23:1977-79 (1958), the entire contents of each of which are incorporated by reference. 5-MeO-DMT may be prepared as the acid addition salt by the addition of any to 5-MeO-DMT that results in a pharmaceutically acceptable salt thereof, including HCl, succinic acid, or sulfuric acid.

### Example 2: Dosing and Safety Data

Disclosed compositions may be prepared comprising from about 4 mg to about 60 mg of 5-MeO-DMT or salt thereof. Disclosed compositions may be formulated for intramuscular injection. The compositions may be administered to a subject having a SUD by intramuscular injection. The total dose administered via intramuscular injection may be from about 6 mg to about 80 mg of 5-MeO-DMT or salt thereof. These compositions may then be used to evaluate pharmacokinetic and pharmacodynamic data in vivo. The pharmacology data can then provide effective dosing ranges, toxicity and modes of action, for the treatment of substance use disorder.

### Example 3: Treatment of Substance Use Disorders

Prior to initial treatment, a subject may undergo preparatory therapy sessions involving psychosocial care. The subject may undergo between 1 to 4 preparatory sessions with a medical practitioner. Optionally, during the preparatory therapy sessions, a subject may be evaluated for a reduction in substance consumption or a reduction in substance abuse as disclosed herein.

After the preparatory therapy sessions, a subject may be evaluated for a reduction in substance consumption or substance abuse over a set period of time as disclosed herein. A subject may be given a score based on decrease in substance use days, a reduction in risk substance level, or a reduction in heavy use days. The evaluation may be done periodically and monitored over several days prior to initial treatment. The subject may undergo initial treatment, wherein the subject is administered 5-MeO-DMT or salt thereof with a medical practitioner. The initial treatment may be administered to a subject based on the subject's score prior to initial treatment.

During the initial treatment session, the subject may be administered a plurality of intramuscular injections. The subject may receive a first intramuscular injection of 6 mg 5-MeO-DMT or salt thereof and be observed by the medical practitioner. The medical practitioner may perform an assessment of the subject after a period of time to elucidate the intensity of the psychotropic effect of the first intramuscular injection of 5-MeO-DMT on the subject. The psychedelic experience may be assessed with the Peak Psychedelic Experience Questionnaire (PPEQ), Peak Experience Scale (PES), the Mystical Experience Questionnaire (MEQ), the Ego Dissolution Inventory (EDI), the Challenging Experience Questionnaire (CEQ), and the 5-Dimensional Altered States of Consciousness Questionnaire (5D-ASC). If the medical practitioner determines that the first intramuscular injection of 5-MeO-DMT did not induce a psychotropic effect of sufficiency in the subject, the medical practitioner may administer an additional intramuscular injection of 5-MeO-DMT. These additional doses of 5-MeO-DMT may be the same amount as the first intramuscular injection (i.e. 6 mg 5-MeO-DMT) or greater. The total dose of 5-MeO-DMT or a salt thereof administered in one injection or a plurality of injections may be up to 80 mg. In some examples, the total dose of 5-MeO-DMT or a salt thereof is between about 25 mg and 50 mg. The observation and administration cycles will continue until the subject has experienced a psychotropic effect of sufficient intensity. At this point, the medical professional will cease administration and observation cycles, and perform a final evaluation of the subject before ending the initial treatment session.

Following initial treatment, the subject will attend a therapy session with a second professional to discuss the subject's experiences during the initial treatment session and any effects on the subject's behavioral patterns and/or mental state after the therapy session. The therapy session occurs between about 1 week and 10 weeks, wherein the range is inclusive, after administration of 5-MeO-DMT or a salt thereof.

The subject's progress can be regularly monitored through regular therapy sessions over a period of between two weeks to eight weeks. Optionally, the subject may monitor their progress using a self-assessment, instructions for which are provided by a medical practitioner. In some cases, the self-assessment can be conducted outside of the regular therapy session.

Additionally, a subject may be administered one or more maintenance treatment sessions after subsequent evaluations by a medical practitioner during regular therapy sessions. The interval between the initial treatment and the maintenance treatment may be at least 1 week and last as long as 12 months. The subject may continue regular therapy sessions at the same, or different, interval from before the administration of a maintenance session.

### Example 4: Comparative Studies

Subjects having a substance use disorder are randomly assigned to a study group, as described below. Prior to treatment, which involves administering 5-MeO-DMT to the subject, the subject may reduce intake of the substance, as confirmed according to known methods.

Provision of Therapy: A pharmaceutical composition comprising 5-MeO-DMT or a salt thereof, e.g., 5-MeO-DMT HCl, is administered to a subject in an amount that does not exceed about 80 mg. The composition may be formulated for sublingual, intravenous, intraarterial, aerosol, parenteral, buccal, topical, transdermal, rectal, intramuscular, subcutaneous, intraosseous, intranasal, intrapulmonary, transmucosal, inhalation, oral, or intraperitoneal administration.

Subjects are assigned to one of three groups: I. Therapy is not provided to the subject after administration of 5-MeO-DMT; II. Therapy is provided to the subject 1 to 6 days after administration of 5-MeO-DMT; III. Therapy is provided to the subject from 1 week to 10 weeks after administration of 5-MeO-DMT, wherein the range is inclusive; IV. Therapy is provided to the subject at least 71 days after administration of 5-MeO-DMT.

Efficacy of the various regimens comprising administration of 5-MeO-DMT and providing psychotherapy, or the absence thereof, in the treatment of the substance use disorder is evaluated by determining i. a reduction in substance intake or substance abstinence, e.g., reduction substance use severity, ii. a reduction in cravings, iii. a reduction in substance cue reactivity, or a combination thereof. Efficacy may also be assessed by measuring changes in biomarkers of stress and inflammation, such as cortisol and IL-6, e.g., as described in Reckweg et al., J Neurochem. 2022 Jul; 162 (1): 128-146 and Uthaug et al., Psychopharmacology (Berl). 2020 Mar;237 (3): 773-785.

Dose: A study having a similar framework to the above is completed to determine the efficacy of a dose of 5-MeO-DMT or a salt thereof in the treatment of a SUD. At least one group of subjects is treated with a total amount of 5-MeO-DMT of up to and including 20 mg, e.g., 1 mg to 20 mg. At least one other group of subjects is treated with a higher dose of 5-MeO-DMT. The higher dose of 5-MeO-DMT is greater than 20 mg, such as about 25 mg or greater. In some examples, the total amount of 5-MeO-DMT is at most 80 mg. The efficacy metrics described above are assessed to determine comparative efficacy. Comparisons are made between groups having the same route of administration, e.g., intramuscular injection or sublingual.

The foregoing description, for purposes of explanation, uses specific nomenclature to provide a thorough understanding of the invention. However, it will be apparent to one skilled in the art that specific details are not required in order to practice the invention. Thus, the foregoing description of specific embodiments of the invention is presented for purposes of illustration and description. It is not intended to be exhaustive or to limit the invention to the precise forms disclosed; obviously, many modifications and variations are possible in view of the above teachings. The embodiments were chosen and described in order to best explain the principles of the invention and its practical applications, through the elucidation of specific examples, and to thereby enable others skilled in the art to best utilize the invention and various embodiments with various modifications as are suited to the particular use contemplated, when such uses are beyond the specific examples disclosed. Accordingly, the scope of the invention shall be defined solely by the following claims and their equivalents.

The invention claimed is:

1. A method of treating a substance use disorder in a subject in need thereof comprising:
a. identifying the subject, wherein the subject has reduced substance consumption for at least 1 day prior to the administration of step (b); and
b. administering to the subject a pharmaceutical composition comprising 5-MeO-DMT or a salt thereof; and
c. providing the subject with a therapy session between 1 week and 10 weeks after the administering of step (b).

2. The method of claim 1, wherein the subject has reduced substance consumption for at least 3, 4, 5, 6, or 7 days prior to step (b).

3. The method of claim 1, wherein the subject has reduced substance consumption for 5-12 days.

4. The method of claim 1, wherein the reduced substance consumption is abstaining from substance consumption.

5. The method of claim 1, wherein the reduced substance consumption is abstaining from moderate to heavy substance consumption.

6. The method of claim 1, wherein the administering of step (b) is performed sublingually, buccally, by intramuscular injection, or intravenous injection.

7. The method of claim 6, wherein the administering of step (b) is performed by an intramuscular injection, or a plurality of intramuscular injections.

8. The method of claim 7, wherein the administering of step (b) is performed by a plurality of intramuscular injections performed within a period of 1 to 8 hours.

9. The method of claim 8, wherein the plurality of intramuscular injections is performed within a period of 2 to 4 hours.

10. The method of claim 6, wherein the total dose of the 5-MeO-DMT or a salt thereof administered in step (b) is between about 6 mg and 80 mg, 10 mg and 80 mg, 20 mg and 80 mg, or 25 mg and 80 mg.

11. The method of claim 10, wherein the total dose of the 5-MeO-DMT or a salt thereof administered in step (b) is between about 25 mg and 50 mg.

12. The method of claim 6, wherein the administering of step (b) is performed by a licensed healthcare professional.

13. The method of any one of claims 1-12, wherein the substance use disorder is alcohol use disorder; cannabis use disorder; hallucinogen use disorder; inhalant use disorder; opioid use disorder; sedatives, hypnotics, or anxiolytics use disorder; stimulant use disorder; or tobacco or nicotine use disorder.

14. The method of claim 13, wherein the substance use disorder is alcohol abuse disorder.

15. The method of claim 13, wherein the substance use disorder is opioid abuse disorder.

16. The method of claim 15, wherein the opioid use disorder comprises abuse of legal prescription opioids.

17. The method of claim 1, wherein the 5-MeO-DMT is an HCl or sulfuric acid addition salt.

18. The method of claim 17, wherein the 5-MeO-DMT is 5-MeO-DMT HCl.

19. The method of claim 1, wherein the therapy session comprises therapy with a licensed professional practitioner and at least one subject.

20. The method of claim 1, wherein the therapy session comprises therapy with a licensed professional practitioner and a plurality of subjects.

21. The method of claim 19 or 21, wherein the therapy session comprises psychosocial therapy.

22. The method of claim 1, wherein the 5-MeO-DMT or salt thereof is not derived from a natural source.

23. The method of claim 22, wherein the 5-MeO-DMT or salt thereof is produced by chemical synthesis.

24. The method of claim 22 or 23, wherein the 5-MeO-DMT comprises less than 5% impurities.

25. The method of claim 24, wherein the 5-MeO-DMT comprises less than 1% of an impurity selected from animal cells, animal tissue, fungal cells, fungal tissue, plant cells, and plant tissue.

26. The method of claim 1, further comprising repeating steps (b) and (c) after a treatment holiday.

27. The method of claim 26, wherein the treatment holiday is between 2 weeks and 2 months.

28. The method of claim 1, wherein administering said treatment attenuates substance intake by said subject by about 20% or more as compared with the amount of substance intake before said treatment.

29. A method of treating a substance use disorder in a subject in need thereof comprising:
a. identifying the subject, wherein the subject has reduced substance consumption for at least 1 day prior to the administration of step (b);
b. administering to the subject a pharmaceutical composition comprising 5-MeO-DMT or a salt thereof; and
c. a treatment holiday of 1 week to 12 months following said administering with the 5-MeO-DMT or salt thereof.

30. The method of claim 29, further comprising providing the subject with a therapy session during the treatment holiday.

31. The method of claim 29, wherein the subject has reduced substance consumption for at least 3, 4, 5, 6, or 7 days prior to step (b).

32. The method of claim 31, wherein the subject has reduced substance consumption for 5-12 days.

33. The method of claim 31, wherein the reduced substance consumption is abstaining from substance consumption.

34. The method of claim 31, wherein the reduced substance consumption is abstaining from moderate to heavy substance consumption.

35. The method of claim 29, wherein the administering of step (b) is performed sublingually, buccally, by intramuscular injection, or intravenous injection.

36. The method of claim 35, wherein the administering of step (b) is performed by an intramuscular injection, or a plurality of intramuscular injections.

37. The method of claim 36, wherein the administering of step (b) is performed by a plurality of intramuscular injections performed within a period of 1 to 8 hours.

38. The method of claim 37, wherein the plurality of intramuscular injections is performed within a period of 2 to 4 hours.

39. The method of claim 35, wherein the total dose of the 5-MeO-DMT or a salt thereof administered in step (b) is between about 6 mg and 80 mg, 10 mg and 80 mg, 20 mg and 80 mg, or 25 mg and 80 mg.

40. The method of claim 39, wherein the total dose of the 5-MeO-DMT or a salt thereof administered in step (b) is between about 25 mg and 50 mg.

41. The method of claim 35, wherein the administering of step (b) is performed by a licensed healthcare professional.

42. The method of any one of claims 29-41, wherein the substance use disorder is alcohol use disorder; cannabis use disorder; hallucinogen use disorder; inhalant use disorder; opioid use disorder; sedatives, hypnotics, or anxiolytics use disorder; stimulant use disorder; tobacco or nicotine use disorder; or a combination thereof.

43. The method of claim 42, wherein the substance use disorder is alcohol abuse disorder.

44. The method of claim 42, wherein the substance use disorder is opioid abuse disorder.

45. The method of claim 44, wherein the opioid use disorder comprises abuse of legal prescription opioids.

46. The method of claim 29, wherein the 5-MeO-DMT is an HCl or sulfuric acid addition salt.

47. The method of claim 46, wherein the 5-MeO-DMT is 5-MeO-DMT HCl.

48. The method of claim 30, wherein the therapy session comprises therapy with a licensed professional practitioner and at least one subject.

49. The method of claim 30, wherein the therapy session comprises therapy with a licensed professional practitioner and a plurality of subjects.

50. The method of claim 48 or 49, wherein the therapy session comprises psychosocial therapy.

51. The method of claim 29, wherein the 5-MeO-DMT or salt thereof is not derived from a natural source.

52. The method of claim 54, wherein the 5-MeO-DMT or salt thereof is produced by chemical synthesis.

53. The method of claim 54 or 55, wherein the 5-MeO-DMT comprises less than 5% impurities.

54. The method of claim 56, wherein the 5-MeO-DMT comprises less than 1% of an impurity selected from animal cells, animal tissue, fungal cells, fungal tissue, plant cells, and plant tissue.

55. The method of claim 55, wherein the treatment holiday is between 2 weeks and 4 months.

56. The method of claim 55, wherein the treatment holiday is between 2 weeks and 2 months.

57. The method of claim 29, further comprising repeating steps (a) and (b) after the treatment holiday.

58. The method of claim 57, further comprising an additional treatment holiday of 1 week to 4 months following repeating steps (a) and (b).

59. A method of treating a substance use disorder in a subject in need thereof comprising:
a. administering a first treatment to the subject, wherein the first treatment comprises administering by a plurality of intramuscular injections a first combined dose of 4 mg to 40 mg of 5-MeO-DMT or a salt thereof; and
b. administering a second treatment to the subject at least four weeks after the first treatment, wherein the second treatment comprises administering by a plurality of intramuscular injections a second combined dose of 5-MeO-DMT or a salt thereof, and wherein the second combined dose is equal to or greater than the first combined dose.

60. The method of claim 59, wherein the method further comprises a first therapy session conducted between the first treatment and the second treatment and a second therapy session conducted after the second treatment.

61. The method of claim 59 or claim 60, wherein the method further comprises one or more maintenance treatments comprising intramuscular injection of 5-MeO-DMT or a salt thereof to the subject and providing a maintenance therapy session after the treatment.

62. The method of claim 61, wherein the first therapy session, the second therapy session, and the maintenance therapy session comprises a practitioner and a plurality of subjects.

63. The method of claim 61, wherein the first therapy session, the second therapy session, and the maintenance therapy session each comprises the practitioner and at least one subject.

64. The method of claim 61, wherein the first therapy session, the second therapy session, and the maintenance therapy session each comprises psychosocial therapy.
